(19) United States
(12) Patent Application Publication
(10) Pub. No.: US 2025/0195574 A1
(43) Pub. Date: Jun. 19, 2025

(54) METHOD FOR REDUCTION OF IMMUNE CHECKPOINT THERAPY RELATED ADVERSE EVENTS

(71) Applicant: NKMAX CO., LTD., Gyeonggi-do (KR)

(72) Inventors: Paul Y. Song, Santa Monica, CA (US); Sang Woo Park, Gyeonggi-do (KR); Yong Man Kim, Gyeonggi-do (KR)

(21) Appl. No.: 18/994,314

(22) PCT Filed: Jun. 1, 2023
(86) PCT No.: PCT/US2023/067766

§ 371 Date: Jan. 14, 2025

Related U.S. Application Data
(60) Provisional application No. 63/348,332, filed on Jun. 2, 2022.

(51) Int. Cl. A61K 35/17; A61K 39/00; A61P 37/06; C07K 16/28

(52) U.S. Cl. CPC A61K 35/17; A61P 37/06; C07K 16/2818; C07K 16/2827; A61K 2039/505; C07K 2317/76

(57) ABSTRACT

The present disclosure is generally related to methods of reducing the frequency and/or severity of adverse events in subjects undergoing checkpoint inhibitor immunotherapy using natural killer (NK) cells.

### INCORPORATION BY REFERENCE TO ANY PRIORITY APPLICATIONS

This application claims the benefit of priority of U.S. Provisional Patent Application No. 63/348,332, filed Jun. 2, 2022, which is hereby expressly incorporated by reference in its entirety, including any appendices filed therewith.

### FIELD

The present disclosure is generally related to methods of reducing the frequency and/or severity of adverse events in subjects undergoing immunotherapy with immune checkpoint inhibitors (ICIs) through the use of natural killer (NK) cells.

### BACKGROUND

Monoclonal antibodies targeting immune checkpoints are able to restore antitumor immunity, thus reversing immune escape or evasion and promoting tumor cell death. Such antibodies include those targeting the cytotoxic T lymphocyte antigen 4 (CTLA-4)-CD28 and programmed cell death 1 (PD-1)-programmed cell death 1 ligand 1 (PD-L1). Treatments utilizing ICIs, such as CTLA-4, PD-1, and/or PD-L1 inhibitors, are associated with adverse events (AEs).

### SUMMARY

The present disclosure is generally related to methods of reducing the frequency and/or severity of adverse events in subjects undergoing immune checkpoint inhibitor (ICI) immunotherapy using natural killer (NK) cells. More specifically, the present disclosure is related to methods of reducing the frequency of adverse events in cancer patients treated with CTLA-4, PD-1, or PD-L1, checkpoint inhibitors using NK cells.

In some embodiments, a method of reducing the likelihood of an adverse event (AE) in a subject treated with an immune checkpoint inhibitor (ICI); the method comprising: administering an ICI to a subject, and administering a collection of natural killer (NK) cells to the subject; wherein NK cells are administered in an amount sufficient to reduce the likelihood and/or severity of one or more AE in the subject is disclosed.

In some embodiments, a method of reducing the likelihood of an adverse event (AE) in a subject treated with an immune checkpoint inhibitor (ICI); the method comprising: identifying a subject at risk of having an AE in response to an ICI, and administering a natural killer (NK) cell to the subject; wherein the adverse event is an immune related adverse event (irAE) is disclosed.

In some embodiments, a method of reducing the likelihood of an adverse event (AE) in a subject treated with an immune checkpoint inhibitor (ICI), the method comprising: identifying a subject who is to, or has received, an ICI; and administering a collection of natural killer (NK) cells to the subject; wherein the adverse event is an immune related adverse event (irAE) is disclosed.

In some embodiments, a method of treating with an ICI comprising: administering an elevated amount of an ICI and an effective amount of NK cells, wherein the effective amount of NK cells is sufficient to reduce at least one AE due to the elevated amount of ICI.

In some embodiments, a method of reducing the likelihood of an adverse event (AE) in a subject treated with an immune checkpoint inhibitor (ICI) is disclosed. In some embodiments, the method comprises administering an ICI to a subject, and administering a collection of natural killer (NK) cells to the subject; wherein NK cells are administered in an amount sufficient to reduce the likelihood and/or severity of one or more AE in the subject as compared to the likelihood and/or severity of the AE in the subject prior to administration of the NK cells.

In some embodiments, a method of reducing the likelihood of an adverse event (AE) in a subject treated with an anti-PD1 or anti-PDL1 therapy is disclosed. In some embodiments, the method comprises: administering an anti-PD1 or anti-PDL1 therapy to a subject, and administering a collection of natural killer (NK) cells to the subject; wherein NK cells are administered in an amount sufficient to reduce the likelihood and/or severity of one or more AE in the subject as compared to the likelihood and/or severity of the AE in the subject prior to administration of the NK cells.

In some embodiments, a method of reducing the likelihood of an adverse event (AE) in a subject treated with pembrolizumab is disclosed. In some embodiments, the method comprises administering pembrolizumab to a subject, and administering CD3−/CD56+ natural killer (NK) cells derived from peripheral blood mononuclear cells (PBMCs) to the subject; wherein the NK cells are administered in an amount sufficient to reduce the likelihood and/or severity of one or more AE in the subject as compared to the likelihood and/or severity of the AE in the subject prior to administration of the NK cells.

In some embodiments, a method of reducing the likelihood of an adverse event (AE) in a subject treated with pembrolizumab is disclosed. In some embodiments, the method comprises administering 200 mg pembrolizumab to a subject, and administering 4×10⁹ administering CD3−/CD56+ natural killer (NK) cells derived from peripheral blood mononuclear cells (PBMCs) to the subject; wherein administration of the NK cells reduces the likelihood and/or severity of one or more AE in the subject as compared to the likelihood and/or severity of the AE in the subject prior to administration of the NK cells.

In some embodiments, the ICI comprises a PD1 inhibitor. In some embodiments, the ICI comprises a PDL1 inhibitor. In some embodiments, the ICI comprises a CTL4 inhibitor. In some embodiments, the ICI comprises a combination PD1 and CTL4 inhibitor.

In some embodiments, the ICI is an ICI selected from the list comprising: pembrolizumab, nivolumab, atezolizomab, durvalumab, avelumab, camrelizumab, cemiplimab, sintilimab, tisleilizumab, toripalimab, lpilimumab, lpilimumab plus nivolumab, or tremelimumab.

In some embodiments, the AE is an AE selected from the list comprising: gastrointestinal toxicity, nausea, vomiting, dysphagia, epigastric pain, abdominal pain, diarrhea, hematochezia, colitis, pneumonitis, rash, other skin AEs, endocrine dysfunction, hepatitis, myocarditis, neurotoxicity, renal failure, hypothyroidism, adrenal insufficiency, nephritis, hypophysitis, diabetes mellitus, myalgia, encephalopathy, irAE, or other ICI related AEs.

In some embodiments, the AE is a serious adverse event (SAE).

In some embodiments, the AE is a treatment emergent adverse event (TEAE).

In some embodiments, the AE, irAE, SAE, or TEAE is a Grade 1, or mild, AE, irAE, SAE, or TEAE.

In some embodiments, the AE, irAE, SAE, or TEAE is a Grade 2, or moderate, AE, irAE, SAE, or TEAE.

In some embodiments, the AE, irAE, SAE, or TEAE is a Grade 3, or severe, AE, irAE, SAE, or TEAE.

In some embodiments, the AE, irAE, SAE, or TEAE is a Grade 4, or life-threatening, AE, irAE, SAE, or TEAE.

In some embodiments, the AE, irAE, SAE, or TEAE is a Grade 5, AE, irAE, SAE, or TEAE.

In some embodiments, the ICI is an anti-cancer ICI. In some embodiments, the ICI is an anti-breast cancer, bladder cancer, cervical cancer, colon cancer, head and neck cancer, Hodgkin lymphoma, liver cancer, lung cancer, non-small cell lung cancer, renal cell cancer, kidney cancer, skin cancer, melanoma, stomach cancer, or rectal cancer ICI.

In some embodiments, the ICI and/or NK cell are administered via parenteral administration, for example, intraperitoneal administration, intravenous administration, intramuscular administration, subcutaneous administration, or intradermal administration.

In some embodiments, the ICI and/or NK cell are administered sequentially.

In some embodiments, the ICI and/or NK cell are administered concurrently.

In some embodiments, the NK cell is an autologous natural killer cell.

In some embodiments, the NK cell expresses NKG2D.

In some embodiments, the NK cell expresses DNAM-1.

In some embodiments, the NK cell expresses NKG2D and DNAM-1.

In some embodiments, each administration of NK cells to the subject comprises administration of between about 2×10⁹ and 4×10⁹ NK cells.

In some embodiments, administration of the NK cells with the ICI reduces the frequency of AEs in subjects by up to about 50%, 90%, or 100% as compared to subjects treated with the ICI alone.

In some embodiments, administration of the NK cells with the ICI reduces the severity of AEs in subjects by between 1-4 Grades.

In some embodiments, administration of the NK cells with the ICI reduces the severity of Grade 5 AEs in subjects by up to about 4 Grades.

In some embodiments, administration of the NK cells with the ICI reduces the severity of Grade 4 AEs in subjects by up to about 3 Grades.

In some embodiments, administration of the NK cells with the ICI reduces the severity of Grade 3 AEs in subjects by up to about 2 Grades.

In some embodiments, administration of the NK cells with the ICI reduces the severity of Grade 2 AEs in subjects by about 1 Grade.

In some embodiments, administration of the NK cells with the ICI increases the progression free survival of subjects as compared to subjects treated with the ICI alone.

In some embodiments, the NK cells limit the survival of auto-reactive CD8 T cells.

In some embodiments, the ICI is administered at about a 10% excess.

In some embodiments, the ICI is administered at about a 20% excess.

In some embodiments, the ICI is administered at about a 30% excess.

In some embodiments, the ICI is administered at about a 40% excess.

In some embodiments, the ICI is administered at about a 50% excess.

In some embodiments, the excess ICI is administered at greater than about a 50% excess.

In some embodiments, the effective amount of administered NK cells is an amount sufficient to reduce the frequency of AEs by up to about 50%, 90%, or 100%, when an ICI is administered at about a 10% excess.

In some embodiments, the effective amount of administered NK cells is an amount sufficient to reduce the frequency of AEs by up to about 50%, 90%, or 100%, when an ICI is administered at about a 20% excess.

In some embodiments, the effective amount of administered NK cells is an amount sufficient to reduce the frequency of AEs by up to about 50%, 90%, or 100%, when an ICI is administered at about a 30% excess.

In some embodiments, the effective amount of administered NK cells is an amount sufficient to reduce the frequency of AEs by up to about 50%, 90%, or 100%, when an ICI is administered at about a 40% excess.

In some embodiments, the effective amount of administered NK cells is an amount sufficient to reduce the frequency of AEs by up to about 50%, 90%, or 100%, when an ICI is administered at about a 50% excess.

In some embodiments, the effective amount of administered NK cells is an amount sufficient to reduce the frequency of AEs by up to about 50%, 90%, or 100%, when an ICI is administered at greater than about a 50% excess.

In some embodiments, the effective amount of administered NK cells is an amount sufficient to reduce the severity of AEs by up to about 1, 2, 3, 4, or 5 Grades when an ICI is administered at about a 10% excess.

In some embodiments, the effective amount of administered NK cells is an amount sufficient to reduce the severity of AEs by up to about 1, 2, 3, 4, or 5 Grades when an ICI is administered at about a 20% excess.

In some embodiments, the effective amount of administered NK cells is an amount sufficient to reduce the severity of AEs by up to about 1, 2, 3, 4, or 5 Grades when an ICI is administered at about a 30% excess.

In some embodiments, the effective amount of administered NK cells is an amount sufficient to reduce the severity of AEs by up to about 1, 2, 3, 4, or 5 Grades when an ICI is administered at about a 40% excess.

In some embodiments, the effective amount of administered NK cells is an amount sufficient to reduce the severity of AEs by up to about 1, 2, 3, 4, or 5 Grades when an ICI is administered at about a 50% excess.

In some embodiments, the effective amount of administered NK cells is an amount sufficient to reduce the severity of AEs by up to about 1, 2, 3, 4, or 5 Grades when an ICI is administered at greater than about a 50% excess.

### BRIEF DESCRIPTION OF THE DRAWINGS

FIG. 1 is an illustration showing embodiments of NK cell NKG2D and DNAM-1 mediated killing. NKG2D and DNAM-1 signaling in the absence of a high inhibitory receptor signal, results in activation of cytolysis and the NK cell releases perforin and granzymes and results in lysis of target cell, for example, an autoreactive Tcell. NKG2D and DNAM-1 are used by NK cells to recognize stress ligands such as, for example, CD155, MIC-A, MIC-B, or ULBP1-6. Chronically activated T cells, including autoreactive T cells, express CD155, MIC family, and ULBP1-6 family ligands.

FIG. 2 is a flow chart showing some embodiments of a method for reducing the frequency of AEs associated with ICI treatment. Item 203 (concurrent administration) is an optional step and, alternatively, the cell can be administered before or after the ICI is administered at separate times, in some embodiments

FIG. 3 is a flow chart showing some embodiments of a method for reducing the frequency of AEs associated with ICI treatment.

FIG. 4 is a flow chart showing some embodiments of a method for reducing the frequency of AEs associated with ICI treatment.

### DETAILED DESCRIPTION

The present disclosure is generally related to methods of reducing the frequency of adverse events (AEs) in subjects undergoing checkpoint inhibitor immunotherapy using natural killer (NK) cells. In some embodiments, the NK cells are Super Natural Killer (SNK) cells. In some embodiments, the NK cells are non-genetically modified and produced from PBMC. In some embodiments, the AE is caused from an ICI therapy and the ICI therapy is an anti-PD1 or anti-PDL1 therapy and the anti-PD1 or anti-PDL1 therapy is optionally an antibody that binds to PD-1 or PDL1.

In some embodiments, a method of reducing the likelihood of an adverse event (AE) in a subject treated with an immune checkpoint inhibitor (ICI) is disclosed. In some embodiments, the method comprises: administering an ICI to a subject and administering a collection of natural killer (NK) cells to the subject. The NK cells are administered in an amount sufficient to reduce the likelihood and/or severity of one or more AE in the subject. In some embodiments, the NK cells are Super Natural Killer (SNK) cells. In some embodiments the NK cells are activated and expanded NK cells. In some embodiments, the NK cells are activated and expanded CD3-CD56+ NK cells using CD56+ and/or CD3-CD56+ NK cells isolated from PBMCs.

In some embodiments, a method of reducing the likelihood of an adverse event (AE) in a subject treated with an immune checkpoint inhibitor (ICI) is disclosed. In some embodiments, the method comprises: identifying a subject at risk of having an AE in response to an ICI and administering a natural killer (NK) cell to the subject. The adverse event is an immune related adverse event (irAE). In some embodiments, the NK cells are Super Natural Killer (SNK) cells. In some embodiments, the NK cells are activated and expanded CD3-CD56+ NK cells using CD56+ and/or CD3-CD56+ NK cells isolated from PBMCs.

In some embodiments, a method of reducing the likelihood of an adverse event (AE) in a subject treated with an immune checkpoint inhibitor (ICI) is disclosed. In some embodiments the method comprises: identifying a subject who is, or has received (or will receive), an ICI and administering a collection of natural killer (NK) cells to the subject. The adverse event is an immune related adverse event (irAE). In some embodiments, the NK cells are Super Natural Killer (SNK) cells. In some embodiments, the NK cells are activated and expanded CD3-CD56+ NK cells using CD56+ and/or CD3-CD56+ NK cells isolated from PBMCs.

In some embodiments, a method of reducing the likelihood and/or severity of an adverse event (AE) in a subject treated with an immune checkpoint inhibitor (ICI) is disclosed. In some embodiments, the method comprises administering an ICI to a subject, and administering a collection of natural killer (NK) cells to the subject; wherein NK cells are administered in an amount sufficient to reduce the likelihood and/or severity of one or more AE in the subject as compared to the likelihood and/or severity of the AE in the subject prior to administration of the NK cells. In some embodiments, the NK cells are Super Natural Killer (SNK) cells. In some embodiments, the NK cells are activated and expanded CD3-CD56+ NK cells using CD56+ and/or CD3-CD56+ NK cells isolated from PBMCs.

In some embodiments, a method of reducing the likelihood of an adverse event (AE) in a subject treated with an anti-PD1 or anti-PD-L1 therapy is disclosed. In some embodiments, the method comprises: administering an anti-PD1 or anti-PD-L1 therapy to a subject, and administering a collection of natural killer (NK) cells to the subject; wherein NK cells are administered in an amount sufficient to reduce the likelihood and/or severity of one or more AE in the subject as compared to the likelihood and/or severity of the AE in the subject prior to administration of the NK cells. In some embodiments, the NK cells are Super Natural Killer (SNK) cells.

In some embodiments, the anti-PD1 therapy comprises nivolumab, pembrolizumab, camrelizumab, cemiplimab, sintilimab, tisleliizumab, zimberelimab, prolgolimab, dostarlimab or toripalimab. In some embodiments, the anti-PD-L1 therapy comprises Atezolizumab (Tecentriq), Avelumab (Bavencio), or Durvalumab (Imfinzi).

In some embodiments, a method of reducing the likelihood of an adverse event (AE) in a subject treated with pembrolizumab is disclosed. In some embodiments, the method comprises administering pembrolizumab to a subject, and administering CD3−/CD56+ natural killer (NK) cells derived from peripheral blood mononuclear cells (PBMCs) to the subject; wherein the NK cells are administered in an amount sufficient to reduce the likelihood and/or severity of one or more AE in the subject as compared to the likelihood and/or severity of the AE in the subject prior to administration of the NK cells. In some embodiments, the NK cells are Super Natural Killer (SNK) cells. In some embodiments, the NK cells are activated and expanded CD3-CD56+ NK cells using CD56+ and/or CD3-CD56+ NK cells isolated from PBMCs.

In some embodiments, a method of reducing the likelihood of an adverse event (AE) in a subject treated with pembrolizumab is disclosed. In some embodiments, the method comprises administering 200 mg pembrolizumab to a subject, and administering 4×10⁹ administering CD3−/CD56+ natural killer (NK) cells derived from peripheral blood mononuclear cells (PBMCs) to the subject; wherein administration of the NK cells reduces the likelihood and/or severity of one or more AE in the subject as compared to the likelihood and/or severity of the AE in the subject prior to administration of the NK cells. In some embodiments, the NK cells are Super Natural Killer (SNK) cells. In some embodiments, the NK cells are activated and expanded CD3-CD56+ NK cells using CD56+ and/or CD3-CD56+NK cells isolated from PBMCs.

### Terminology

As used herein, the term “immune checkpoint inhibitors” (ICIs) has its plain and ordinary meaning as read in light of the specification and may refer to a type of drug that blocks protein checkpoints. Protein checkpoints may be made by some types of immune system cells, such as T cells, and some cancer cells. These protein checkpoints help keep immune responses from being too strong and sometimes can keep T cells from killing cancer cells. When these protein checkpoints are blocked, T cells can kill cancer cells better. Non-limiting examples of checkpoint proteins found on T cells or cancer cells include PD-1/PD-L1 and CTLA-4/B7-1/B7-2. Some immune checkpoint inhibitors are used to treat cancer.

As used herein, the term “adverse event” (AE) has its plain and ordinary meaning as read in light of the specification and may refer to an undesired effect of a drug or other type of treatment, such as surgery. Adverse events can range from mild to severe and can be life-threatening. The term “grade” may refer to the severity of the AE. A grade 1 AE may refer to a mild AE. A grade 2 AE may refer to a moderate AE. A grade 3 may refer to a severe AE. A grade 4 AE may refer to a life-threatening or disabling AE. A grade 5 AE may refer to a death related to AE. An AE may also be referred to as an “adverse effect” and “adverse reaction.”

As used herein, the term “immune related adverse event” (AE) has its plain and ordinary meaning as read in light of the specification and may refer to adverse events that occurred in the context of exposure to an immunotherapeutic drug and are consistent with the development of an autoimmune reaction, and are not attributable to another cause (e.g., infection, trauma, other drugs).

As used herein the term “natural killer cell” (NK cell) has its plain and ordinary meaning as read in light of the specification and may refer to a type of innate immune cells, which are known to non-specifically kill cancer, recognize and kill virus-infected cells, bacteria, and the like, and kill pathogens with enzymes such as perforin and granzyme or by Fas-FasL interaction. In the case of cancer patients, it has been reported that a decrease in cancer cell cytotoxicity of these NK cells is associated with the onset of various types of cancer, such as lung cancer (Carrega P, et al., Cancer, 2008: 112: 863-875), liver cancer (Jinushi M, et al., J Hepatol., 2005: 43; 1013-1020), breast cancer (Bauernhofer T, et al., Eur J Immunol. 2003: 33; 119-124), uterine cancer (Mocchegiani E., et al., Br j Cancer., 1999: 79: 244-250), blood cancer (Tajima F., et al, Leukemia 1996: 10:478-482), and the like. Accordingly, for cancer therapy, it is desirable Jo increase the cancer cell cytotoxicity of the NK cells.

As used herein, the term “Super Natural Killer cell” (SNK cell) has its plain and ordinary meaning as read in light of the specification and may refer to a type of NK cell displaying superior cell killing ability at lower ratios as compared to NK cells. SNK cells may be included in cell collections at a high purity. SNK cells may proliferate from 1,000 to 19 billion fold. In some embodiments, the SNK cells are CD3−/CD56+ SNK cells.

As used herein, the terms “treating” or “treatment”, and related terms such as “inhibiting”, “reducing”, and “preventing”, (and as well understood in the art) means an approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delaying or slowing of disease progression, amelioration or palliation of the disease state, diminishment of the reoccurrence of disease, and remission, whether partial or total and whether detectable or undetectable. As provided herein, “preventing” may include one or more of “inhibiting” or “reducing”. “Treating” and “treatment” as used herein may include a therapeutic treatment (e.g., in response to a current disease state) and/or a prophylactic treatment (e.g. to prevent the occurrence or severity of an anticipated disease state). Treatment methods comprise administering to a subject a therapeutically effective amount of an active agent. The administering step may consist of a single administration or may comprise a series of administrations. The compositions are administered to the subject in an amount and for a duration sufficient to treat the subject. The length of the treatment period depends on a variety of factors, such as the severity of the condition, the age and genetic profile of the subject, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required.

The terms “effective amount” or “effective dose” as used herein have their plain and ordinary meaning as understood in light of the specification, and refer to that amount of a recited composition or compound that results in an observable designated effect. Actual dosage levels of active ingredients in an active composition of the presently disclosed subject matter can be varied so as to administer an amount of the active composition or compound that is effective to achieve the designated response for a particular subject and/or application. The selected dosage level can vary based upon a variety of factors including, but not limited to, the activity of the composition, formulation, route of administration, combination with other drugs or treatments, severity of the condition being treated, and the physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are contemplated herein.

In some non-limiting embodiments, an effective amount or effective dose of a composition or compound may relate to the amount or dose that provides a significant, measurable, or sufficient therapeutic effect towards the treatment of any one or more of the diseases provided herein, such as cardiovascular diseases, for example, stroke, traumatic brain injury, cerebral amyloid angiopathy, atherosclerosis, myocardial infarction, and/or diseases associated with fibrin activity or dysfunction, or cancers. In some embodiments, the effective amount or effective dose of a composition or compound may treat, ameliorate, or prevent the progression of symptoms of any one or more of the diseases provided herein.

The term “administering” includes oral administration, topical contact, administration as a suppository, intravenous, intraperitoneal, intramuscular, intralesional, intrathecal, intranasal, or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a first compound described herein is administered at the same time, just prior to, or just after the administration of a second compound described herein.

As used herein, the term “therapeutic target” refers to a gene or gene product that, upon modulation of its activity (e.g., by modulation of expression, biological activity, and the like), can provide for modulation of the disease phenotype. As used throughout, “modulation” is meant to refer to an increase or a decrease in the indicated phenomenon (e.g., modulation of a biological activity refers to an increase in a biological activity or a decrease in a biological activity).

As used herein, “pharmaceutically acceptable” has its plain and ordinary meaning as understood in light of the specification and refers to carriers, excipients, and/or stabilizers that are nontoxic to the cell or mammal being exposed thereto at the dosages and concentrations employed or that have an acceptable level of toxicity. A “pharmaceutically acceptable” “diluent,” “excipient,” and/or “carrier” as used herein have their plain and ordinary meaning as understood in light of the specification and are intended to include any and all solvents, dispersion media, coatings, antibacterial and antifungal agents, isotonic and absorption delaying agents, and the like, compatible with administration to humans, cats, dogs, or other vertebrate hosts. Typically, a pharmaceutically acceptable diluent, excipient, and/or carrier is a diluent, excipient, and/or carrier approved by a regulatory agency of a Federal, a state government, or other regulatory agency, or listed in the U.S. Pharmacopeia or other generally recognized pharmacopeia for use in animals, including humans as well as non-human mammals, such as cats and dogs. The term diluent, excipient, and/or carrier can refer to a diluent, adjuvant, excipient, or vehicle with which the pharmaceutical formulation is administered. Such pharmaceutical diluent, excipient, and/or carriers can be sterile liquids, such as water and oils, including those of petroleum, animal, vegetable or synthetic origin. Water, saline solutions and aqueous dextrose and glycerol solutions can be employed as liquid diluents, excipients, and/or carriers, particularly for injectable solutions. Suitable pharmaceutical diluents and/or excipients include sugars, starch, glucose, fructose, lactose, sucrose, maltose, gelatin, malt, rice, flour, chalk, silica gel, sodium stearate, glycerol monostearate, talc, salts, sodium chloride, dried skim milk, glycerol, propylene, glycol, water, ethanol and the like. A non-limiting example of a physiologically acceptable carrier is an aqueous pH buffered solution. The physiologically acceptable carrier may also comprise one or more of the following: antioxidants, such as ascorbic acid, low molecular weight (less than about 10 residues) polypeptides, proteins, such as serum albumin, gelatin, immunoglobulins, hydrophilic polymers such as polyvinylpyrrolidone, amino acids, carbohydrates such as glucose, mannose, or dextrins, chelating agents such as EDTA, sugar alcohols such as glycerol, erythritol, threitol, arabitol, xylitol, ribitol, mannitol, sorbitol, galactitol, fucitol, iditol, inositol, isomalt, maltitol, or lactitol, salt-forming counterions such as sodium, and nonionic surfactants such as TWEEN®, polyethylene glycol (PEG), and PLURONICS®. The formulation, if desired, can also contain minor amounts of wetting, bulking, emulsifying agents, or pH buffering agents. These formulations can take the form of solutions, suspensions, emulsion, sustained release formulations and the like. The formulation should suit the mode of administration.

As used herein, the term “pharmaceutically acceptable salts” has its plain and ordinary meaning as understood in light of the specification and includes relatively non-toxic, inorganic and organic acid, or base addition salts of compositions or excipients, including without limitation, analgesic agents, therapeutic agents, other materials, and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc, and the like. Salts may also be formed with suitable organic bases, including those that are non-toxic and strong enough to form such salts. For example, the class of such organic bases may include but are not limited to mono-, di-, and trialkylamines, including methylamine, dimethylamine, and triethylamine; mono-, di-, or trihydroxyalkylamines including mono-, di-, and triethanolamine; amino acids, including glycine, arginine and lysine; guanidine; N-methylglucosamine; N-methylglucamine; L-glutamine; N-methylpiperazine; morpholine; ethylenediamine; N-benzylphenethylamine; trihydroxymethyl aminoethane.

As used herein, a “carrier” refers to a compound, particle, solid, semi-solid, liquid, or diluent that facilitates the passage, delivery and/or incorporation of a compound to cells, tissues and/or bodily organs. For example, without limitation, a lipid nanoparticle (LNP) is a type of carrier that can encapsulate an oligonucleotide to thereby protect the oligonucleotide from degradation during passage through the bloodstream and/or to facilitate delivery to a desired organ, such as to the lungs.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

The term “excipient” has its ordinary meaning as understood in light of the specification, and refers to inert substances, compounds, or materials added to a pharmaceutical composition to provide, without limitation, bulk, consistency, stability, binding ability, lubrication, disintegrating ability etc., to the composition. Excipients with desirable properties include but are not limited to preservatives, adjuvants, stabilizers, solvents, buffers, diluents, solubilizing agents, detergents, surfactants, chelating agents, antioxidants, alcohols, ketones, aldehydes, ethylenediaminetetraacetic acid (EDTA), citric acid, salts, sodium chloride, sodium bicarbonate, sodium phosphate, sodium borate, sodium citrate, potassium chloride, potassium phosphate, magnesium sulfate sugars, dextrose, dextran, fructose, mannose, lactose, galactose, sucrose, sorbitol, cellulose, methyl cellulose, hydroxypropyl methyl cellulose (hypromellose), glycerin, polyvinyl alcohol, povidone, propylene glycol, serum, amino acids, polyethylene glycol, polysorbate 20, polysorbate 80, sodium deoxycholate, sodium taurodeoxycholate, magnesium stearate, octylphenol ethoxylate, benzethonium chloride, thimerosal, gelatin, esters, ethers, 2-phenoxyethanol, urea, or vitamins, or any combination thereof. The amount of the excipient may be found in a pharmaceutical composition at a percentage of 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 95%, 100% w/w or any percentage by weight in a range defined by any two of the aforementioned numbers.

Additional excipients with desirable properties include but are not limited to preservatives, adjuvants, stabilizers, solvents, buffers, diluents, solubilizing agents, detergents, surfactants, chelating agents, antioxidants, alcohols, ketones, aldehydes, ethylenediaminetetraacetic acid (EDTA), tris(hydroxymethyl)aminomethane (Tris), citric acid, ascorbic acid, acetic acid, salts, phosphates, citrates, acetates, succinates, chlorides, bicarbonates, borates, sulfates, sodium chloride, sodium bicarbonate, sodium phosphate, sodium borate, sodium citrate, potassium chloride, potassium phosphate, magnesium sulfate sugars, dextrose, dextran 40, fructose, mannose, lactose, trehalose, galactose, sucrose, sorbitol, mannitol, cellulose, serum, amino acids, alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, polysorbate 20, polysorbate 40, polysorbate, 60, polysorbate 80, poloxamer, poloxamer 188, sodium deoxycholate, sodium taurodeoxycholate, magnesium stearate, octylphenol ethoxylate, benzethonium chloride, thimerosal, gelatin, esters, ethers, 2-phenoxyethanol, urea, or vitamins, or any combination thereof. Some excipients may be in residual amounts or contaminants from the process of manufacturing, including but not limited to serum, albumin, ovalbumin, antibiotics, inactivating agents, formaldehyde, glutaraldehyde, β-propiolactone, gelatin, cell debris, nucleic acids, peptides, amino acids, or growth medium components or any combination thereof. The amount of the excipient may be found in the formulation at a percentage that is at least 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 95%, 100% w/w or any percentage by weight in a range defined by any two of the aforementioned numbers.

The term “purity” of any given substance, compound, or material as used herein refers to the actual abundance of the substance, compound, or material relative to the expected abundance. For example, the substance, compound, or material may be at least 80, 85, 90, 91, 92, 93, 94, 95, 96, 97, 98, 99, or 100% pure, including all decimals in between. Purity may be affected by unwanted impurities, including but not limited to side products, isomers, enantiomers, degradation products, solvent, carrier, vehicle, or contaminants, or any combination thereof. Purity can be measured technologies including but not limited to chromatography, liquid chromatography, gas chromatography, spectroscopy, UV-visible spectrometry, infrared spectrometry, mass spectrometry, nuclear magnetic resonance, gravimetry, or titration, or any combination thereof.

As used herein, the term “standard of care”, “best practice” and “standard therapy” refers to the treatment that is accepted by medical practitioners to be an appropriate, proper, effective, and/or widely used treatment for a certain disease. The standard of care of a certain disease depends on many different factors, including the biological effect of treatment, region or location within the body, patient status (e.g. age, weight, gender, hereditary risks, other disabilities, secondary conditions), toxicity, metabolism, bioaccumulation, therapeutic index, dosage, and other factors known in the art. Determining a standard of care for a disease is also dependent on establishing safety and efficacy in clinical trials as standardized by regulatory bodies such as the US Food and Drug Administration, International Council for Harmonisation, Health Canada, European Medicines Agency, Therapeutics Goods Administration, Central Drugs Standard Control Organization, National Medical Products Administration, Pharmaceuticals and Medical Devices Agency, Ministry of Food and Drug Safety, and the World Health Organization. The standard of care for a disease may include but is not limited to surgery, radiation, chemotherapy, targeted therapy, or immunotherapy.

As used herein, the term “inhibit” refers to the reduction or decrease in an expected activity, such as a cellular activity. The reduction or decrease may be by at least 5%, 10%, 20%, 30%, 40%, 50%, 60%, 70%, 80%, 90%, or 100%, or any percentage that is within a range defined by any two of the aforementioned values, where a reduction or decrease of 100% indicates a complete inhibition and any lower percentage indicates a partial inhibition. The reduction or decrease of the expected activity may be observed in a direct or indirect way.

Immunotherapy with immune checkpoint inhibitors (ICIs) has had an enormous impact on the treatment of diverse types of cancer, leading to durable remissions in a subset of patients and significantly extending survival for others.

Some broadly effective types of these therapies are monoclonal antibodies that block the immune checkpoints cytotoxic T lymphocyte antigen (CTLA)-4, programmed death (PD)-1 or its ligand PD-L1. Alongside the tremendous clinical benefit of immunotherapy has come a diverse array of inflammatory toxicities or adverse events (AEs) that can affect any organ system in the body. These toxicities are an important cause of morbidity, frequently lead to treatment discontinuation, and can have debilitating long-term consequences. As used herein, the term AE means any unfavorable and unintended sign (including an abnormal laboratory finding), symptom, or disease temporally associated with use of an ICI, whether or not related to ICI. A serious adverse event (SAE) means an AE that results in any of following outcomes: death; life threatening; persistent/significant disability/incapacity; initial or prolonged inpatient hospitalization; congenital anomaly/birth defect or was otherwise considered medically important. Treatment-emergent adverse events (TEAE) are events between first dose of study drug that were absent before treatment or that worsened relative to pre-treatment state up to 30 days after last administration. TEAEs include both Serious TEAEs and non-serious TEAEs. The severity of TEAEs can be graded using National Cancer Institute-Common Terminology Criteria for Adverse Events (NCI-CTCAE) version 4.0 toxicity grades, as follows: Grade 1=Mild, Grade 2=Moderate, Grade 3=Severe, Grade 4=Life-threatening and Grade 5=Death.

Because ICIs activate T immune cells in a variety of tissues, they are often associated with autoimmune side effects, termed “immune related adverse events” (irAEs). These irAEs commonly affect the skin, colon, liver, lungs, endocrine organs, and joints, but can affect almost any organ. IrAEs can mimic autoimmune diseases seen in other settings, but often differ at the level of tissue pathology. The term “T immune cell,” or “T cell,” refers to a lymphocyte derived from thymus, which can “memorize” previously encountered antigens and provide information to B cells, thereby facilitates production of antibody and plays an important role in cell immune system. Since these T cells may distinguish very small differences among different antigens to induce an immune response to allogenic antigens, autologous therapy is possible, but there may be a limit to be used for allogenic therapy.

The incidence of irAEs varies depending on the ICI regimen used. The best estimates come from large systematic reviews and meta-analyses. The incidence of any irAEs with anti-CTLA-4 antibodies is estimated to be 72% and of high grade irAEs 24%. Patients treated with anti-PD-1 or anti-PD-L1 antibodies have a similar total incidence of irAEs, 74%, but fewer high-grade irAEs, 14%.

ICIs block inhibitory checkpoint and activate I-cell mediated immune response. The precise mechanism of irAEs is still unknown, but several hypotheses are suggested. The key idea is that the use of ICIs breaks up the immunologic homeostasis and reduces T-cell tolerance. irAEs are mediated by the development of autoimmunity through release of the surviving autoreactive CD8 T cells, generation of pre-existing autoreactive antibodies, or on-target attack of normal tissue expressing shared tumor antigens.

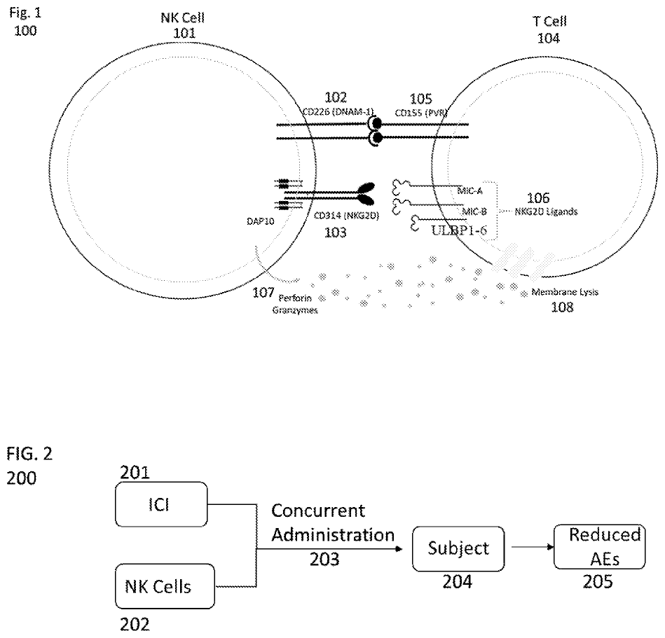

FIG. 1 is an illustration showing embodiments of NK cell NKG2D and DNAM-1 mediated killing.

Natural Killer (NK) Cells 101 are large granular lymphocytes that express multiple activation and inhibitory receptors. These receptors allow NK cells to rapidly survey their environment for danger. When an imbalance in signaling favors activation, secretion of cytokines and/or release of cytotoxic granules occur. Activated NK cells also kill immature dendritic cells (DC) thus regulating T cell expansion and decreasing the risk of proinflammatory tissue damage. NK cells can directly limit the survival of activated reactive T cells through, for example, NKG2D and DNAM-1 expression. NKG2D (CD314) 102 is a surface receptor that is expressed by all NK cells and transmits an activating signal via the DAP10 adaptor molecule 103. DNAM-1 (CD226) 102 is a cell surface glycoprotein that functions as an adhesion molecule to synergize with activating receptors and trigger NK cell mediated cytotoxicity upon interaction with its ligands CD155 (PVR) and CD112. NKG2D and DNAM-1 signaling in the absence of high inhibitory receptor signal, results in activation of cytolysis and the NK cell releases performing and granzymes 107, and lysis of the target cell 108, in this case an autoreactive T cell 104. NKG2D and DNAM-1 are used by NK cells to recognize stress ligands. Chronically activated T cells, including autoreactive T cells, express CD155, Mic family ligands including Mic-A, Mic-B, and Mic-C, and ULBP family ligands. In some embodiments, the NK cells are activated and expanded CD3-CD56+ NK cells using CD56+ and/or CD3-CD56+ NK cells isolated from PBMCs.

In some embodiments, a method of reducing the likelihood of an adverse event (AE) in a subject treated with an immune checkpoint inhibitor (ICI) is disclosed. In some embodiments, the method comprises: administering an ICI to a subject, and administering a collection of natural killer (NK) cells to the subject; wherein NK cells are administered in an amount sufficient to reduce the likelihood and/or severity of one or more AE in the subject.

FIG. 2 is a flow chart showing some embodiments of a method for reducing the likelihood of AEs associated with ICI treatment. In some embodiments, the ICI therapy is an anti-PD1 or anti-PDL1 therapy and is optionally an antibody that binds to PD-1 or PDL1.

In some embodiments, a method 200 for reducing the frequency of AEs associated with ICI treatment is disclosed. In some embodiments, the AE is an AE, SAE, irAE, or TEAE. In some embodiments, the AE comprises gastrointestinal toxicity, nausea, vomiting, dysphagia, epigastric pain, abdominal pain, diarrhea, hematochezia, colitis, pneumonitis, rash, other skin AEs, endocrine dysfunction, hepatitis, myocarditis, neurotoxicity, renal failure, hypothyroidism, adrenal insufficiency, nephritis, hypophysitis, diabetes mellitus, myalgia, encephalopathy, irAE, or other ICI related AEs. In some embodiments the ICI is a CTLA-4, PD-1, and/or PD-L1 ICI. In some embodiments the ICI 201 is administered to a subject. In some embodiments, the subject is a mammal. In some embodiments, the subject is a human. In some embodiments, NK cells 202 are administered to a subject. In some embodiments, the NK cells comprise autologous NK cells. In some embodiments, the NK cells comprise non-autologous NK cells. In some embodiments, the NK cells are whole-blood derived NK cells. In some embodiments, the NK cells are peripheral blood derived NK cells. In some embodiments, the NK cells are derived from umbilical cord blood or from iPSCs. In some embodiments, the ICI and NK cells are administered to the subject concurrently (203, optional). In some embodiments, the ICI and NK cells are administered sequentially. In some embodiments, NK cells are administered concurrently with each dose of ICI. In some embodiments, NK cells are administered sequentially before or after each dose of ICI. In some embodiments, administration of NK cells with an ICI results in decreased frequency of AEs 205. In some embodiments, the NK cells are activated and expanded CD3-CD56+ NK cells using CD56+ and/or CD3-CD56+ NK cells isolated from PBMCs.

In some embodiments, a method of reducing the likelihood and/or severity of an adverse event (AE) in a subject treated with an immune checkpoint inhibitor (ICI) is disclosed. In some embodiments, the method comprises: identifying a subject at risk of having an AE in response to an ICI, and administering a natural killer (NK) cell to the subject. The adverse event is an immune related adverse event (irAE).

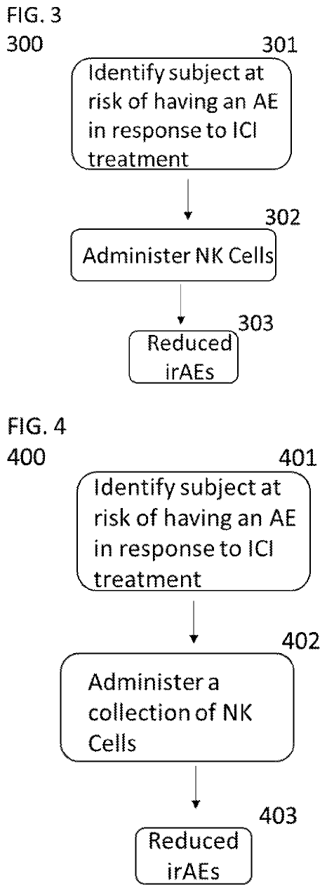

FIG. 3 is a flow chart showing some embodiments of a method for reducing the frequency of AEs associated with IIC treatment.

In some embodiments, a method 300 for reducing the frequency of irAEs associated with ICI treatment is disclosed. In some embodiments the ICI is a CTLA-4, PD-1, and/or PD-L1 ICI. In some embodiments the ICI is administered to a subject. In some embodiments, the subject is a mammal. In some embodiments, the subject is a human. Subjects at risk of developing an AE are identified 301. In some embodiments, administration results in an AE, SAE, irAE, or TEAE. In some embodiments, the subject is identified as being at risk for an AE, SAE, irAE, or TEAE prior to ICI treatment. In some embodiments, the AE, SAE, irAE, or TEAE, is identified after administration of an ICI. In some embodiments, NK cells 302 are administered to a subject. In some embodiments, the NK cells comprise autologous NK cells. In some embodiments, the NK cells comprise non-autologous cells. In some embodiments, the NK cells are whole-blood derived NK cells. In some embodiments, the NK cells are peripheral blood derived NK cells. In some embodiments, the NK cells are derived from umbilical cord blood or from iPSCs. In some embodiments, the ICI and NK cells are administered to the subject concurrently. In some embodiments, the ICI and NK cells are administered sequentially. In some embodiments, NK cells are administered concurrently with each dose of ICI. Administration of NK cells with an ICI results in decreased frequency of irAEs 303. In some embodiments, the NK cells are activated and expanded CD3-CD56+ NK cells using CD56+ and/or CD3-CD56+ NK cells isolated from PBMCs.

FIG. 4 is a flow chart showing some embodiments of a method for reducing the frequency of AEs associated with IIC treatment.

In some embodiments, a method of reducing the likelihood of an adverse event (AE) in a subject treated with an immune checkpoint inhibitor (ICI) is disclosed. In some embodiments the method comprises: identifying a subject who is, or has received, an ICI; administering a collection of natural killer (NK) cells to the subject; wherein the adverse event is an immune related adverse event (irAE).

In some embodiments, a method of reducing the likelihood of an AE in a subject treated with an ICI is disclosed. In some embodiments, the ICI comprises CTLA-4, PD-1, and/or PD-1L. In some embodiments the ICI is administered to a subject. In some embodiments, the subject is a mammal. In some embodiments, the subject is a human. The subject is identified as being at risk of an ICI related AE 401. In some embodiments, administration of the ICI results in an AE, SAE, irAE, or TEAE. In some embodiments, the subject is identified as being at risk for an AE, SAE, irAE, or TEAE prior to ICI treatment. In some embodiments, the AE, SAE, irAE, or TEAE, is identified after administration of an ICI. In some embodiments, a collection of NK cells 402 are administered to a subject. In some embodiments, the NK cells comprise autologous NK cells. In some embodiments, the NK cells comprise non-autologous cells. In some embodiments, the NK cells are whole-blood derived NK cells. In some embodiments, the NK cells are peripheral blood derived NK cells. In some embodiments, the NK cells are derived from umbilical cord blood or from iPSCs. In some embodiments, the ICI and NK cells are administered to the subject concurrently. In some embodiments, the ICI and NK cells are administered sequentially. In some embodiments, NK cells are administered concurrently with each dose of ICI. In some embodiments, NK cells are administered sequentially after each dose of ICI. Administration of NK cells with an ICI results in decreased frequency of irAEs 403. In some embodiments, the NK cells are activated and expanded CD3-CD56+ NK cells using CD56+ and/or CD3-CD56+ NK cells isolated from PBMCs.

In some embodiments, the ICI is a PD-1 inhibitor. In some embodiments, the PD-1 inhibitor comprises nivolumab, pembrolizumab, camrelizumab, cemiplimab, sintilimab, tisleilizumab, or toripalimab. In some embodiments, the ICI is a CTLA-4 inhibitor. In some embodiments, the CTLA-4 inhibitor is lpilimumab or tremelimumab. In some embodiments, the ICI is a PD-L1 inhibitor. In some embodiments, the PD-L1 inhibitor is atezolizumab, avelumab, or durvalumab. In some embodiments, the anti-PD1 or anti-PDL1 therapy is an antibody that binds to PD-1 or PDL1.

In some embodiments the ICI is an ICI selected from the list comprising: pembrolizumab, nivolumab, atezolizomab, durvalumab, avelumab, camrelizumab, cemiplimab, sintilimab, tisleiliizumab, toripalimab, lpilimumab, zimberelimab, prolgolimab, lpilimumab plus nivolumab, or tremelimumab. In some embodiments, the ICI is a combination PD1 and CTL4 inhibitor. In some embodiments, the ICI is a commercially available ICI. A non-exhaustive list of commercially available ICIs is shown in Table 1

TABLE 1

| Checkpoint inhibitor Therapies | Companies |
|---|---|
| Pembrolizumab | Merck |
| Nivolumab | BMS |
| Atezolizomab | Roche |
| Durvalumab | AstraZeneca |
| Avelumab | Pfizer Merck KGaA |
| Camrelizumab | Jiangsu HengRui |
| Cemiplimab | Regeneron |
| Sintilimab | Innovent Eli Lilly |
| Tislelizumab | Beigene |
| Toripalimab | Junshi Biosscience |
| Ipilimumab | BMS |
| Tremelimumab | MedImmune |

In some embodiments the AE is an AE selected from the list comprising: gastrointestinal toxicity, nausea, vomiting, dysphagia, epigastric pain, abdominal pain, diarrhea, hematochezia, colitis, pneumonitis, rash, other skin AEs, endocrine dysfunction, hepatitis, myocarditis, neurotoxicity, renal failure, hypothyroidism, adrenal insufficiency, nephritis, hypophysitis, diabetes mellitus, myalgia, encephalopathy, irAE, or other ICI related AEs.

In some embodiments, the ICI is an anti-cancer ICI. In some embodiments, the ICI is an anti-breast cancer, bladder cancer, cervical cancer, colon cancer, head and neck cancer, Hodgkin lymphoma, liver cancer, lung cancer, non-small cell lung cancer, renal cell cancer, kidney cancer, skin cancer, melanoma, stomach cancer, or rectal cancer ICI. In some embodiments, the ICI is an anti-solid tumor ICI.

In some embodiments, the ICI and/or NK cell are administered via parenteral administration, for example, intraperitoneal administration, intravenous administration, intramuscular administration, subcutaneous administration, or intradermal administration.

In some embodiments, the NK cells can be derived from a blood sample. As used herein, the “blood sample” may be, but not limited to, whole blood of the peripheral blood or leukocytes isolated from the peripheral blood using leukapheresis. In some embodiments, the NK cells are derived from umbilical cord blood or from iPSCs. Further, the peripheral blood may be obtained from a normal person, a patient having a risk of cancer, or a cancer patient, but the source of the peripheral blood is not limited thereto.

In some embodiments, the NK cell is an autologous natural killer cell. In some embodiments, the NK cell is a peripheral blood derived cell. The term “peripheral blood-derived” may mean that the cells are derived from “whole blood of the peripheral blood” or “leukocytes isolated from the peripheral blood using leukapheresis.” In some embodiments, the NK cell is a peripheral blood mononuclear cell. The term “peripheral blood mononuclear cell” may be used interchangeably with “PBMC” or “mononuclear cell,” and may refer to a mononuclear cell isolated from the peripheral blood which is generally used for anti-cancer immunotherapy. The peripheral blood mononuclear cells may be obtained from the collected human blood using known methods such as a Ficoll-Hypaque density gradient method. In some embodiments, the NK cells are activated and expanded CD3-CD56+ NK cells using CD56+ and/or CD3-CD56+ NK cells isolated from PBMCs.

As used herein, the term “leukapheresis” may refer to a method of selectively removing (isolating) leukocytes from the collected blood and then giving the blood to a patient again, and in some embodiments, isolated leukocytes may be used without additional methods such as a Ficoll-Hypaque density gradient method.

In some embodiments, the peripheral blood mononuclear cells may be autologous, but allogenic peripheral blood mononuclear cells may also be used for producing high-purity NK cells for anti-cancer immunotherapy. Further, in some embodiments, the peripheral blood mononuclear cells may be obtained from a normal person, but the peripheral blood mononuclear cells may be also obtained from a patient having a risk of cancer and/or a cancer patient.

In some embodiments, the NK cell is a CD56+/CD3− cell. The CD3−/CD56+ cells may include cells in which CD56 glycoprotein on the cell surface is expressed, or further, cells in which CD3 glycoprotein is not expressed while the CD56 glycoprotein is expressed. Even the same type of immune cells may have differences in CD type attached to the cell surface and expression rate and thus, the functions thereof may be different. In some embodiments, the NK cell expresses NKG2D. In some embodiments, the NK cell expresses DNAM-1. In some embodiments, the NK cell expresses NKG2D and DNAM-1. In some embodiments, the NK cell expresses CD16, CD25, CD27, CD28, CD69, CD94/NKG2C, CD94/NKG2E, CD266, CD244, KIR2S, KIR3S, Ly94D, NCRs, IFN-a, IFN-b, CXCR3, CXCR4, CX3CR1, CD62L or CD57.

In some embodiments, the NK cells are produced by isolating peripheral blood mononuclear cells (PBMCs) from a blood sample (First Isolation Step); isolating at least one of CD56+ cells and/or CD3−/CD56+ cells from the PBMCs (Second Isolation Step); and co-culturing the at least one of CD56+ cells and/or CD3−/CD56+ cells with a combination of “feeder cells” in the presence of a cytokine (Culturing Step). As used herein, the term feeder cells refers to a cell or cells that do not divide and proliferate, but have metabolic activity to produce various metabolites and thus, helps the proliferation of target cells.

In some embodiments, CD56+ cells and CD3−/CD56+ cells are isolated from PBMCs using a Ficoll-Hypaque density gradient method and then counting the cells. In some embodiments, the counted PBMCs are added with a MACS buffer (1×PBS+0.5% HSA) and suspended, and added with CD56 microbeads (Miltenyi Biotec) to be 1 to 20 μL per 1.0×10⁷ PBMCs, and then incubated at 2 to 8° C. for about 5 to 30 minutes. In some embodiments, after incubation, the MACS buffer is added and mixed, and then the mixture is centrifuged (600×g) to precipitate the cells. After centrifugation, the supernatant may be removed, and the cells suspended by adding the MACS buffer. The suspension is then added in a column connected to a MACS separator. The MACS buffer may be passed through the column to remove non-specific binding. In some embodiments, the column is separated from the MACS separator and transferred to a 15 mL conical tube, and then added with the MACS buffer to isolate CD56+ cells attached to the column.

In some embodiments, CD56+ cells and CD3−/CD56+ cells are isolated from PBMCs using a Ficoll-Hypaque density gradient method and then counting the cells. In some embodiments, the counted PBMCs are added with a MACS buffer (1×PBS±0.5% HSA) and suspended. The suspension is then added with CD3 microbeads (Miltenyi Biotec) to be about 1 to 20 μL per 1.0×10⁷ PBMCs, and then incubated at 2 to 8° C. for 5 to 30 minutes. After incubation, the MACS buffer is added and mixed, and then the mixture is centrifuged (600×g) to precipitate the cells. After centrifugation, a supernatant is removed, and the cells are suspended by adding the MACS buffer. The suspension is then added in a column connected to a MACS separator. The MACS buffer may be passed through the column to collect CD3− cells. The collected CD3− cells are then added with a MACS buffer (1×PBS+0.5% HSA) and suspended, and added with CD56 microbeads (Miltenyi Biotec) to be about 1 to 20 μL per 1.0×10⁷ CD3− cells, and then incubated at 2 to 8° C. for 5 to 30 minutes. After incubation, the MACS buffer is added and mixed, and then the mixture is centrifuged (600×g) to precipitate the cells. After centrifugation, a supernatant is removed, and the cells are suspended by adding the MACS buffer. The suspension is then added in a column connected to a MACS separator. The MACS buffer is passed through the column to remove non-specific binding. The column is separated from the MACS separator and transferred to a 15 mL conical tube, and then added with the MACS buffer to isolate CD3−/CD56+ cells attached to the column.

In some embodiments, CD56+ cells or the CD3−/CD56+ cells isolated from PBMCs are added in a RPMI-1640 medium containing FBS 10% added with IL-2 at a concentration of 500 IU/mL together with prepared combination of feeder cells (Jurkat cells and EBV-LCL cells) irradiated with 100 Gy radiation and then co-cultured in an incubator at 37° C. and 5% CO2. In some embodiments, CD56+ cells or the CD3−/CD56+ cells isolated from PBMCs are added in a RPMI-1640 medium containing FBS 10% added with IL-2 (500 IU/mL) and IL-21 (50 ng/mL) together with prepared combination of feeder cells (Jurkat cells and EBV-LCL cells) irradiated with 100 Gy radiation and then co-cultured in an incubator at 37° C. and 5% CO2. In some embodiments, the ratio of (CD56+ cells and/or CD3−/CD56+ cells):(Jurkat cells):(EBV-LCL cells) is about 1:30:30. In some embodiments, the Jurkat cells may be obtained from ATCC (ATCC TIB-152), and the EBV-LCL cells may be prepared by the following method: 30×10⁶ PBMCs is added in 9 mL of a culture medium, the mixture is added in a T 25 culture flask, and then 9 mL of an EBV supernatant is added. 80 μL of cyclosporine A is added and then cultured at 37° C. After 7 days of culture, a half of supernatant is removed, a fresh culture medium is added, and then 40 μL of cyclosporine A is added. The same process as the 7th day is repeated once every 7 days until 28 days of culture. The cell line is usable after 28 days of culture, and from this time, the cell line is cultured in the culture medium without adding cyclosporine A.

In some embodiments, PBMCs are isolated from the blood using a Ficoll-Hypaque density gradient method. In some embodiments, the PBMCs are then added in a RPMI-1640 medium containing FBS 10% added with IL-2 at a concentration of 500 IU/mL together with prepared feeder cells (Jurkat cells and EBV-LCL cells) irradiated with 100 Gy radiation and then co-cultured in an incubator at 37° C. and 5% CO2.

In some embodiments, PBMCs are isolated from the blood using a Ficoll-Hypaque density gradient method. In some embodiments, the PBMCs are then added in a RPMI-1640 medium containing FBS 10% added with IL-2 (500 IU/mL) and IL-21 (50 ng/mL) together with prepared feeder cells (Jurkat cells and EBV-LCL cells) irradiated with 100 Gy radiation and then co-cultured in an incubator at 37° C. and 5% CO2.

In some embodiments, the SNK are derived from PBMCs that were isolated from the blood using a Ficoll-Hypaque density gradient method. In some embodiments, the PBMCs are then added in a RPMI-1640 medium containing FBS 10% added with IL-2 (500 IU/mL) and IL-21 (50 ng/mL) together with prepared feeder cells (Jurkat cells and EBV-LCL cells at a ratio of 1:0.5:0.5) irradiated with 100 Gy radiation and then co-cultured in an incubator at 37° C. and 5% CO2.

In some embodiments, the reduction in frequency of an AE, or irAE, correlates to the amount of NK cells administered. In some embodiments, the reduction in frequency of an AR, or irAE, is constant above a threshold dosage of NK cells.

In some embodiments, of NK cells administered is about 1×10⁶, 2.5×10⁶, 5×10⁶, 7.5×10⁶, 1×10⁷, 2.5×10⁷, 5×10⁷, 7.5×10⁷, 1×10⁸, 2.5×10⁸, 5×10⁸, 7.5×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2.5×10¹⁰, 5×10¹⁰, 7.5×10¹⁰, 1×10¹¹, 2.5×10¹¹, 5×10¹¹, 7.5×10¹¹ NK cells, or is a range that is defined by any two of the preceding values. For example, in some embodiments, each administration of NK cells to the subject comprises administration of between about 1×10⁶ and 1×10¹¹, 1×10⁶ and 1×10¹⁰, 1×10⁶ and 9×10⁹, 1×10⁶ and 9×10⁸, 1×10⁶ and 9×10⁷, 1×10⁷ and 9×10⁹, 1×10⁸ and 9×10⁹, 1×10⁹ and 9×10⁹, 1×10⁹ and 6×10⁹, or 2×10⁹ and 4×10⁹ NK cells. In some embodiments, about 1×10⁶, 2.5×10⁶, 5×10⁶, 7.5×10⁶, 1×10⁷, 2.5×10⁷, 5×10⁷, 7.5×10⁷, 1×10⁸, 2.5×10⁸, 5×10⁸, 7.5×10⁸, 9×10⁸NK cells per kg of body weight are administered, or a range that is defined by any two of the preceding values. For example, in some embodiments, between about 1×10⁶ and 9×10⁸, 1×10⁶ and 9×10⁷, 1×10⁷ and 9×10⁸, 1×10⁸ and 9×10⁸ NK cells per kg of body weight are administered.

In some embodiments, administration of the NK cells in combination with the ICI reduces the frequency of AEs in subjects by up to about 1%, 2%, 3%, 4%, 5%, 6%, 7%, 8%, 9%, 10%, 15%, 20%, 25%, 30%, 35%, 40%, 45%, 50%, 60%, 70%, 80%, 90%, 95%, 96%, 97%, 98%, 99%, or 100% as compared to subjects treated with the ICI alone, or is a range that is defined by any two of the preceding values. For example, in some embodiments, administration of the NK cells with the ICI reduces the frequency of AEs in subjects by between about 1-50%, 1-40%, 1-30%, 1-25%, 1-20%, 1-15%, 1-10%, 1-5%, 5-50%, 5-40%, 5-30%, 5-25%, 10-50%, 10-40%, 10-30%, or 10-20%.

Some exemplary frequencies of various AEs associated with ICI monotherapy and ICI+NK cells are listed in Table 6.

In some embodiments, the frequency of diarrhea is reduced by about 1%, 2%, 3%, 4%, 5%, 6%, 7%, 8%, 9%, 10%, 11%, 12%, 13%, 14%, 15%, 16%, 17%, 18%, 19%, 19.1% 20%, 25%, 30%, 35% 40%, 50%, 60%, 70%, 80%, 90%, or 100% or is a range that is defined by any two of the preceding values. For example, in some embodiments the frequency of diarrhea is reduced by between about 1%-40%, 1%-30%, 1%-20%, 1%-19.1%, 1%-15%, 1%-10%, 1-6%, 5%-40%, 5%-30%, 5%-25%, or 5%-20%.

In some embodiments, the frequency of colitis is reduced by about 1%, 2%, 3%, 3.1%, 3.2%, 3.3%, 3.4%, 3.5%, 3.6%, 3.7%, 3.8%, 3.9%, 4%, 5%, 6%, 7%, 8%, 9%, 10%, 11%, 12%, 13%, 14%, 15%, 16%, 17%, 18%, 19%, 19.1% 20%, 25%, 30%, 35%, 40%, 50%, 60%, 70%, 80%, 90%, or 100%, or is a range that is defined by any two of the preceding values. For example, in some embodiments the frequency of colitis is reduced by between about 1%-40%, 1%-30%, 1%-20%, 1-15%, 1%-10%, 1%-4%, 1%-3.7%, 5%-40%, 5%-30%, 5%-25%, 5%-20%, or 3.7%-20%.

In some embodiments, the frequency of pneumonitis is reduced by about 1%, 2%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 6%, 7%, 8%, 9%, 10%, 15%, 20%, 30%, 40%, 50%, 60%, 70%, 80%, 90%, or 100% or is a range that is defined by any two of the preceding values. For example, in some embodiments the frequency of pneumonitis is reduced by between about 1%-20%, 1%-10%, 1%-5%, or 1-4.7%.

In some embodiments, the frequency of skin adverse events are reduced by about 1%, 2%, 3%, 4%, 5%, 6%, 7%, 8%, 9%, 10%, 11%, 12%, 13%, 14%, 15%, 16%, 16.1%, 16.2%, 16.3%, 16.4%, 16.5%, 16.6%, 16.7%, 16.8%, 16.9% 17%, 18%, 19%, 19.1% 20%, 25%, 30%, 35%, 40%, 50%, 60%, 70%, 80%, 90%, or 100% or is a range that is defined by any two of the preceding values. For example, in some embodiments the frequency of skin adverse events are reduced by between about 1%-40%, 1%-30%, 1%-20%, 1%-16.1%, 1%-15%, 1%-13%, 1%-10%, 1-7%, 5%-40%, 5%-30%, 5%-25%, or 5%-20%.

In some embodiments, the frequency of endocrine dysfunction is reduced by about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 23.1%, 23.2%, 23.3%, 23.4%, 23.5%, 23.6%, 23.7%, 23.8%, 23.9%, 24%, 25%, 30%, 35%, 40%, 50%, 60%, 70%, 80%, 90%, or 100% or is a range that is defined by any two of the preceding values. For example, in some embodiments the frequency of endocrine dysfunction is reduced by between about 1%-50%, 1%-40%, 1%-30%, 1%-25%, 1%-23.40%, 1%-20%, 1%-15%, 1%-10%, 1-7%, 5%-50%, 5%-40%, 5%-30%, 5%-25%, or 5%-20%.

In some embodiments, the frequency of hepatitis is reduced by about 1%, 1.1%, 1.2%, 1.3%, 1.4%, 1.5%, 1.6%, 1.7%, 1.8%, 1.9% 2%, 3%, 4%, 5%, 6%, 7%, 8%, 9%, 10%, 15%, 20%, 30%, 40%, 50%, 60%, 70%, 80%, 90%, or 100% or is a range that is defined by any two of the preceding values. For example, in some embodiments the frequency of diarrhea is reduced by between about 1%-20%, 1%-10%, 1%-5%, or 1-2%.

In some embodiments, the frequency of renal failure is reduced by about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 50%, 60%, 70%, 80%, 90%, or 100% or is a range that is defined by any two of the preceding values. For example, in some embodiments the frequency of renal failure is reduced by between about 1%-40%, 1%-30%, 1%-20%, 1%-19.1%, 1%-15%, 1%-10%, 1-6%, 5%-40%, 5%-30%, 5%-25%, or 5%-20%.

In some embodiments, the frequency of irAEs are reduced by about 1%, 2%, 3%, 4%, 5%, 6%, 7%, 8%, 9%, 10%, 15%, 16%, 16.5%, 20%, 25%, 30%, 35%, 40%, 45%, 47%, 50%, 55%, 60%, 65%, 69%, 70%, 75%, 80%, 85%, 90%, 95%, 100%, or by a range that is defined by any two of the preceding values. For example, in some embodiments, the frequency of irAEs is reduced by between about 1%-95%, 1%-80%, 1%-75%, 1%-69%, 1%-50%, 1%-47%, 1%-30%, 1%-25%, 1%-16.5%, 10%-95%, 10%-80%, 10%-75%, 10%-69%, 10%-50%, 10%-47%, or 16.5%-69%.

In some embodiments, the NK cells are administered concurrently with the ICI. In some embodiments, administration of the NK cells concurrently (optionally, or serially) with the ICI reduces the frequency of AEs in subjects by up to about 1%, 2%, 3%, 4%, 5%, 6%, 7%, 8%, 9%, 10%, 15%, 20%, 25%, 30%, 35%, 40%, 50%, 60%, 70%, 80%, 90%, or 100% as compared to subjects treated with the ICI alone, or is a range that is defined by any two of the preceding values. For example, in some embodiments, administration of the NK cells concurrently with the ICI reduces the frequency of AEs in subjects by between about 1-50%, 1-40%, 1-30%, 1-25%, 1-20%, 1-15%, 1-10%, 1-5%, 5-50%, 5-40%, 5-30%, 5-25%, 10-50%, 10-40%, 10-30%, or 10-20%.

In some embodiments, in the case of an adult, the NK cells can be administered once to several times a day. In some embodiments, the NK cells are administered for 1, 2, 3, 4, 5, 6, 7, 10, 14, 21, 28, 29, 30, 31, 45, 60, 90, 120, 150, or 180-days, or in an interval that is defined by any two of the preceding values. For example, in some embodiments, the NK cells are administered for 1-180, 1-150, 1-120, 1-90, 1-60, 1-45, 1-31, 1-30, 1-29, 1-28, 1-21, 1-14, 1-7, 1-5, 1-3, 7-180, 7-120, 7-90, 7-45, 7-30, 7-14, 7-10, 30-180, 30-150, 30-120, 30-90, 30-60, or 30-45 days.

TEAEs are the events between first dose of study drug that were absent before treatment or that worsened relative to pre-treatment. Thus, in some embodiments, the present methods can involve the use of NK cells for reducing, preventing, and or minimizing one or more TEAEs. In some embodiments, the events arise or worsen for 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days after last administration, or for a range that is defined by any two of the preceding values. For example, in some embodiments, the TEAEs arise between about 1-30, 1-28, 1-21, 1-15, 1-14, 1-10, 1-7, 1-5, 1-3, 7-30, 7-28, 7-21, 7-14, 15-30, or 15-20 days after last administration of the ICI. In some embodiments, administration of NK cells with an ICI results in decreased frequency of TEAEs. In some embodiments, administration of the ICI to subjects with an AE or TEAE would be ceased in the absence of NK cell treatment.

In some embodiments, the severity of the AE, SAE, irAE, or TEAE is a Grade 1, Grade 2, Grade 3, Grade 4, or Grade 5 AE, SAE, irAE, or TEAE. In some embodiments, the subject experiences more than 1 AE, SAE, irAE, or TEAE. In some embodiments, the AEs, SAEs, irAEs, or TEAEs, are of the same grade. In some embodiments, the AEs, SAEs, irAEs, or TEAEs, are of a different grade. In some embodiments, administration of the NK cells to the subject reduces the severity of the AE, SAE, irAE, or TEAE, by 1, 2, 3, 4, or 5 grades. In some embodiments, administration of the NK cells to the subject reduces the severity of the AE, SAE, irAE, or TEAE, from Grade 5 to Grade 4, Grade 3, Grade 2, Grade 1, or eliminates the AE, SAE, irAE, or TEAE altogether. In some embodiments, administration of the NK cells to the subject reduces the severity of the AE, SAE, irAE, or TEAE, from Grade 4 to Grade 3, Grade 2, Grade 1, or eliminates the AE, SAE, irAE, or TEAE altogether. In some embodiments, administration of the NK cells to the subject reduces the severity of the AE, SAE, irAE, or TEAE, from Grade 3 to Grade 2, Grade 1, or eliminates the AE, SAE, irAE, or TEAE altogether. In some embodiments, administration of the NK cells to the subject reduces the severity of the AE, SAE, irAE, or TEAE, from Grade 2 to Grade 1, or eliminates the AE, SAE, irAE, or TEAE altogether. In some embodiments, administration of the NK cells to the subject reduces the severity of all AEs, SAEs, irAEs, or TEAEs, by the same amount, or number of grades. In some embodiments, In some embodiments, administration of the NK cells to the subject reduces the severity of all AEs, SAEs, irAEs, or TEAEs, by a different amount, or number of grades. In some embodiments, administration of the cells reduces 1, 2, 3, 4, 5, 6, 7, 8, 9 or more TEAEs (such as one or more of those in Table 6). In some embodiments, any of the above TEAE aspects can be with respect to those options listed in table 6.

In some embodiments, the amount of ICI administered is greater than, or in excess of, the amount of ICI that would be administered in the absence of administration NK cells. In some embodiments, the ICI is administered in about a 1%, 2%, 3%, 4%, 5%, 10%, 15%, 20%, 25%, 30%, 35%, 40%, 45%, 50%, 60%, 70%, 75%, 80%, 90%, 91%, 92%, 93%, 94%, 95%, 96%, 97%, 98%, 99%, or 100% excess, or is a range that is defined by any two of the preceding values. For example, in some embodiments, the ICI is administered in between about 1% and 100%, 1% and 75%, 1% and 50%, 1% and 25%, 1% and 10%, 10% and 100%, 10% and 75%, 10% and 50%, 10% and 25%, 25% and 100%, 25% and 75%, or 25% and 50% excess. In some embodiments, the ICI is administered at greater than about a 100% excess. For example, in some embodiments, the ICI is administered at a 1-fold, 2-fold, 3-fold, 4-fold, 5-fold, 6-fold, 7-fold, 8-fold, 9-fold, or 10-fold excess, or in a range that is defined by any two of the preceding values. Fore example, in some embodiments, the ICI is administered at between 1-fold and 10-fold, 1-fold and 5-fold, 1-fold and 3-fold, 3-fold and 10-fold, 3-fold and 7-fold, 5-fold and 10-fold, or 5-fold and 7-fold excess.

In some embodiments, NK cells are administered at an effective amount to reduce the frequency and/or severity of one or more AEs when an ICI is administered in excess (where excess is compared to the current standard amount of administration, in the absence of the NK cells). In some embodiments, the effective amount of NK cells administered is sufficient to reduce the frequency of AEs by up to about 1%, 5%, 10%, 15%, 20%, 25%, 30%, 40%, 50%, 60%, 70%, 80%, 90%, or 100%, when an ICI is administered at about a 10% excess or is a range that is defined by any two of the preceding values. For example, in some embodiments, the effective amount of NK cells administered is sufficient to reduce the frequency of AEs by between about 1%-100%, 1%-75%, 1%-50%, 1%-25%, 5%-100%, 5%-75%, 5%-50%, 5%-25%, 25%-100%, 25%-75%, 25%-50%, 50%-100%, 50%-75%, or 75%-100%.

In some embodiments, the effective amount of NK cells administered is sufficient to reduce the frequency of AEs by up to about 1%, 5%, 10%, 15%, 20%, 25%, 30%, 40%, 50%, 60%, 70%, 80%, 90%, or 100%, when an ICI is administered at about a 20% excess or is a range that is defined by any two of the preceding values. For example, in some embodiments, the effective amount of NK cells administered is sufficient to reduce the frequency of AEs by between about 1%-100%, 1%-75%, 1%-50%, 1%-25%, 5%-100%, 5%-75%, 5%-50%, 5%-25%, 25%-100%, 25%-75%, 25%-50%, 50%-100%, 50%-75%, or 75%-100%.

In some embodiments, the effective amount of NK cells administered is sufficient to reduce the frequency of AEs by up to about 1%, 5%, 10%, 15%, 20%, 25%, 30%, 40%, 50%, 60%, 70%, 80%, 90%, or 100%, when an ICI is administered at about a 30% excess or is a range that is defined by any two of the preceding values. For example, in some embodiments, the effective amount of NK cells administered is sufficient to reduce the frequency of AEs by between about 1%-100%, 1%-75%, 1%-50%, 1%-25%, 5%-100%, 5%-75%, 5%-50%, 5%-25%, 25%-100%, 25%-75%, 25%-50%, 50%-100%, 50%-75%, or 75%-100%.

In some embodiments, the effective amount of NK cells administered is sufficient to reduce the frequency of AEs by up to about 1%, 5%, 10%, 15%, 20%, 25%, 30%, 40%, 50%, 60%, 70%, 80%, 90%, or 100%, when an ICI is administered at about a 40% excess or is a range that is defined by any two of the preceding values. For example, in some embodiments, the effective amount of NK cells administered is sufficient to reduce the frequency of AEs by between about 1%-100%, 1%-75%, 1%-50%, 1%-25%, 5%-100%, 5%-75%, 5%-50%, 5%-25%, 25%-100%, 25%-75%, 25%-50%, 50%-100%, 50%-75%, or 75%-100%.

In some embodiments, the effective amount of NK cells administered is sufficient to reduce the frequency of AEs by up to about 1%, 5%, 10%, 15%, 20%, 25%, 30%, 40%, 50%, 60%, 70%, 80%, 90%, or 100%, when an ICI is administered at about a 50% excess or is a range that is defined by any two of the preceding values. For example, in some embodiments, the effective amount of NK cells administered is sufficient to reduce the frequency of AEs by between about 1%-100%, 1%-75%, 1%-50%, 1%-25%, 5%-100%, 5%-75%, 5%-50%, 5%-25%, 25%-100%, 25%-75%, 25%-50%, 50%-100%, 50%-75%, or 75%-100%.

In some embodiments, the effective amount of NK cells administered is sufficient to reduce the frequency of AEs by up to about 1%, 5%, 10%, 15%, 20%, 25%, 30%, 40%, 50%, 60%, 70%, 80%, 90%, or 100%, when an ICI is administered at greater than about a 50% excess, or is a range that is defined by any two of the preceding values. For example, in some embodiments, the effective amount of NK cells administered is sufficient to reduce the frequency of AEs by between about 1%-100%, 1%-75%, 1%-50%, 1%-25%, 5%-100%, 5%-75%, 5%-50%, 5%-25%, 25%-100%, 25%-75%, 25%-50%, 50%-100%, 50%-75%, or 75%-100%.

In some embodiments, NK cells are administered at an effective amount to reduce the frequency and/or severity of one or more AEs when an ICI is administered in excess. In some embodiments, the effective amount of NK cells administered is sufficient to reduce the severity of AEs by up to 1, 2, 3, 4, or 5 grades when an ICI is administered at about a 10% excess or is a range that is defined by any two of the preceding values. For example, in some embodiments, the effective amount of NK cells administered is sufficient to reduce the severity of AEs by between about 1-5, 1-4, 1-3, 1-2, 2-5, 2-4, 2-3, 3-5, 3-4, or 4-5 grades when an ICI is administered at about a 10% excess.

In some embodiments, the effective amount of NK cells administered is sufficient to reduce the severity of AEs by up to 1, 2, 3, 4, or 5 grades when an ICI is administered at about a 10% excess or is a range that is defined by any two of the preceding values. For example, in some embodiments, the effective amount of NK cells administered is sufficient to reduce the severity of AEs by between about 1-5, 1-4, 1-3, 1-2, 2-5, 2-4, 2-3, 3-5, 3-4, or 4-5 grades when an ICI is administered at about a 20% excess.

In some embodiments, the effective amount of NK cells administered is sufficient to reduce the severity of AEs by up to 1, 2, 3, 4, or 5 grades when an ICI is administered at about a 10% excess or is a range that is defined by any two of the preceding values. For example, in some embodiments, the effective amount of NK cells administered is sufficient to reduce the severity of AEs by between about 1-5, 1-4, 1-3, 1-2, 2-5, 2-4, 2-3, 3-5, 3-4, or 4-5 grades when an ICI is administered at about a 30% excess.

In some embodiments, the effective amount of NK cells administered is sufficient to reduce the severity of AEs by up to 1, 2, 3, 4, or 5 grades when an ICI is administered at about a 10% excess or is a range that is defined by any two of the preceding values. For example, in some embodiments, the effective amount of NK cells administered is sufficient to reduce the severity of AEs by between about 1-5, 1-4, 1-3, 1-2, 2-5, 2-4, 2-3, 3-5, 3-4, or 4-5 grades when an ICI is administered at about a 40% excess.

In some embodiments, the effective amount of NK cells administered is sufficient to reduce the severity of AEs by up to 1, 2, 3, 4, or 5 grades when an ICI is administered at about a 10% excess or is a range that is defined by any two of the preceding values. For example, in some embodiments, the effective amount of NK cells administered is sufficient to reduce the severity of AEs by between about 1-5, 1-4, 1-3, 1-2, 2-5, 2-4, 2-3, 3-5, 3-4, or 4-5 grades when an ICI is administered at about a 50% excess.

In some embodiments, the effective amount of NK cells administered is sufficient to reduce the severity of AEs by up to 1, 2, 3, 4, or 5 grades when an ICI is administered at about a 10% excess or is a range that is defined by any two of the preceding values. For example, in some embodiments, the effective amount of NK cells administered is sufficient to reduce the severity of AEs by between about 1-5, 1-4, 1-3, 1-2, 2-5, 2-4, 2-3, 3-5, 3-4, or 4-5 grades when an ICI is administered at greater than about a 50% excess.

In some embodiments, the methods disclosed herein further comprise administration of a cytokine. The term “cytokine” can refer to an immuno-active compound that is usable to induce the peripheral blood mononuclear cells to differentiate into NK cells. In some embodiments, the cytokine may be interleukin-2 (IL-2), IL-15, IL-21, FMS-like tyrosine kinase 3 ligand (Flt3-L), a stem cell factor (SCF), IL-7, IL-18, IL-4, type I interferons, a granulocyte-macrophage colony-stimulating factor (GM-CSF), and an insulin-like growth factor 1 (IGF 1), but not limited thereto. In some embodiments, the cytokine may be used at a concentration of 50-1,000, 50-900, 50-800, 50-700, 50-600, 50-550, 100-550, 150-550, 200-550, 250-550, 300-550, 350-550, 400-550, 450-550 IU/mL. When the cytokine is used in these ranges, it may suppress apoptosis of the NK cells included in the cancer treatment composition and increase anti-cancer activity of the NK cells.

In some embodiments, administration of the NK cells concurrently with the ICI increases the progression free survival of subjects as compared to subjects treated with the ICI alone. In some embodiments, the NK cells limit the survival of autoreactive CD8 T cells.

### Numbered Arrangements 1:

Some embodiments provided herein are described by way of the following provided numbered arrangements and also provided as possible combinations or overlapping embodiments:

1. A method of reducing the likelihood of an adverse event (AE) in a subject treated with an immune checkpoint inhibitor (ICI); the method comprising:
administering an ICI to a subject, and
administering a collection of natural killer (NK) cells to the subject; wherein
   NK cells are administered in an amount sufficient to reduce the likelihood and/or severity of one or more AE in the subject.

2. A method of reducing the likelihood of an adverse event (AE) in a subject treated with an immune checkpoint inhibitor (ICI); the method comprising:
identifying a subject at risk of having an AE in response to an ICI, and
administering a natural killer (NK) cell to the subject; wherein the adverse event is an immune related adverse event (irAE).

3. A method of reducing the likelihood of an adverse event (AE) in a subject treated with an immune checkpoint inhibitor (ICI), the method comprising:
identifying a subject who is to, or has received, an ICI; and
administering a collection of natural killer (NK) cells to the subject; wherein
the adverse event is an immune related adverse event (irAE).

4. A method of treating a subject with an ICI comprising: administering 1) an elevated amount of an ICI and 2) an effective amount of NK cells, wherein the effective amount of NK cells is sufficient to reduce at least one AE due to the elevated amount of ICI.

5. The method of any of the preceding arrangements, wherein the ICI comprises a PD1 inhibitor.

6. The method of any of the preceding arrangements, wherein the ICI comprises a PDL1 inhibitor.

7. The method of any of the preceding arrangements, wherein the ICI comprises a CTL4 inhibitor.

8. The method of any of the preceding arrangements, wherein the ICI comprises a combination PD1 and CTL4 inhibitor.

9. The method of any of the preceding arrangements, wherein the ICI is an ICI selected from the list comprising: pembrolizumab, nivolumab, atezolizomab, durvalumab, avelumab, camrelizumab, cemiplimab, sintilimab, tisleilizumab, toripalimab, lpilimumab, lpilimumab plus nivolumab, or tremelimumab.

10. The method of any of the preceding arrangements, wherein the AE is an AE selected from the list comprising: gastrointestinal toxicity, nausea, vomiting, dysphagia, epigastric pain, abdominal pain, diarrhea, hematochezia, colitis, pneumonitis, rash, other skin AEs, endocrine dysfunction, hepatitis, myocarditis, neurotoxicity, renal failure, hypothyroidism, adrenal insufficiency, nephritis, hypophysitis, diabetes mellitus, myalgia, encephalopathy, irAE, or other ICI related AEs.

11. The method of any of the preceding arrangements, wherein the AE is a serious adverse event (SAE).

12. The method of any of the preceding arrangements, wherein the AE is a treatment emergent adverse event (TEAE).

13. The method of any of the preceding arrangements, wherein the AE, irAE, SAE, or TEAE is a Grade 1, or mild, AE, irAE, SAE, or TEAE.

14. The method of any of the preceding arrangements, wherein the AE, irAE, SAE, or TEAE is a Grade 2, or moderate, AE, irAE, SAE, or TEAE.

15. The method of any of the preceding arrangements, wherein the AE, irAE, SAE, or TEAE is a Grade 3, or severe, AE, irAE, SAE, or TEAE.

16. The method of any of the preceding arrangements, wherein the AE, irAE, SAE, or TEAE is a Grade 4, or life-threatening, AE, irAE, SAE, or TEAE.

17. The method of any of the preceding arrangements, wherein the AE, irAE, SAE, or TEAE is a Grade 5, AE, irAE, SAE, or TEAE.

18. The method of any of the preceding arrangements, wherein the ICI is an anti-cancer ICI.

19. The method of any of the preceding arrangements, wherein the ICI is an anti-breast cancer, bladder cancer, cervical cancer, colon cancer, head and neck cancer, Hodgkin lymphoma, liver cancer, lung cancer, non-small cell lung cancer, renal cell cancer, kidney cancer, skin cancer, melanoma, stomach cancer, or rectal cancer ICI.

20. The method of any of the preceding arrangements, wherein the ICI and/or NK cell are administered via parenteral administration, for example, intraperitoneal administration, intravenous administration, intramuscular administration, subcutaneous administration, or intradermal administration.

21. The method of any of the preceding arrangements, wherein the ICI and/or NK cell are administered sequentially.

22. The method of any of the preceding arrangements, wherein the ICI and/or NK cell are administered concurrently.

23. The method of any of the preceding arrangements, wherein the NK cell is an autologous natural killer cell.

24. The method of any of the preceding arrangements, wherein the NK cell expresses NKG2D.

25. The method of any of the preceding arrangements, wherein the NK cell expresses DNAM-1.

26. The method of any of the preceding arrangements, wherein the NK cell expresses NKG2D and DNAM-1.

27. The method of any of the preceding arrangements, wherein each administration of NK cells to the subject comprises administration of between about 2×10⁹ and 4×10⁹ NK cells.

28. The method of any of the preceding arrangements, wherein administration of the NK cells with the ICI reduces the frequency of AEs in subjects by up to about 50%, 90%, or 100% as compared to subjects treated with the ICI alone.

29. The method of any of the preceding arrangements, wherein administration of the NK cells with the ICI reduces the severity of AEs in subjects by between 1-4 Grades.

30. The method of any of the preceding arrangements, wherein administration of the NK cells with the ICI reduces the severity of Grade 5 AEs in subjects by up to about 4 Grades.

31. The method of any of the preceding arrangements, wherein administration of the NK cells with the ICI reduces the severity of Grade 4 AEs in subjects by up to about 3 Grades.

32. The method of any of the preceding arrangements, wherein administration of the NK cells with the ICI reduces the severity of Grade 3 AEs in subjects by up to about 2 Grades.

33. The method of any of the preceding arrangements, wherein administration of the NK cells with the ICI reduces the severity of Grade 2 AEs in subjects by about 1 Grade.

34. The method of any of the preceding arrangements, wherein administration of the NK cells with the ICI increases the progression free survival of subjects as compared to subjects treated with the ICI alone.

35. The method of any of the preceding arrangements, wherein the NK cells limit the survival of auto-reactive CD8 T cells.

36. the method of any of the preceding arrangements, wherein the ICI is administered at about a 10% excess (compared to an amount that is the current standard of care, in an absence of NK cells).

37. The method of any of the preceding arrangements, wherein the ICI is administered at a 20% excess (compared to an amount that is the current standard of care, in an absence of NK cells).

38. The method of any of the preceding arrangements, wherein the ICI is administered at about a 30% excess (compared to an amount that is the current standard of care, in an absence of NK cells).

39. The method of any of the preceding arrangements, wherein the ICI is administered at about a 40% excess (compared to an amount that is the current standard of care, in an absence of NK cells).

40. The method of any of the preceding arrangements, wherein the ICI is administered at about a 50% excess (compared to an amount that is the current standard of care, in an absence of NK cells).

41. The method of any of the preceding arrangements, wherein the excess ICI is administered at greater than about a 50% excess (compared to an amount that is the current standard of care, in an absence of NK cells).

42. The method of any of the preceding arrangements, wherein the effective amount of administered NK cells is an amount sufficient to reduce the frequency of AEs by up to about 50%, 90%, or 100%, when an ICI is administered at about a 10% excess (compared to an amount that is the current standard of care, in an absence of NK cells).

43. The method of any of the preceding arrangements, wherein the effective amount of administered NK cells is an amount sufficient to reduce the frequency of AEs by up to about 50%, 90%, or 100%, when an ICI is administered at about a 20% excess (compared to an amount that is the current standard of care, in an absence of NK cells).

44. The method of any of the preceding arrangements, wherein the effective amount of administered NK cells is an amount sufficient to reduce the frequency of AEs by up to about 50%, 90%, or 100%, when an ICI is administered at about a 30% excess (compared to an amount that is the current standard of care, in an absence of NK cells).

45. The method of any of the preceding arrangements, wherein the effective amount of administered NK cells is an amount sufficient to reduce the frequency of AEs by up to about 50%, 90%, or 100%, when an ICI is administered at about a 40% excess (compared to an amount that is the current standard of care, in an absence of NK cells).

46. The method of any of the preceding arrangements, wherein the effective amount of administered NK cells is an amount sufficient to reduce the frequency of AEs by up to about 50%, 90%, or 100%, when an ICI is administered at about a 50% excess (compared to an amount that is the current standard of care, in an absence of NK cells).

47. The method of any of the preceding arrangements, wherein the effective amount of administered NK cells is an amount sufficient to reduce the frequency of AEs by up to about 50%, 90%, or 100%, when an ICI is administered at greater than about a 50% excess (compared to an amount that is the current standard of care, in an absence of NK cells).

48. The method of any of the preceding arrangements, wherein the effective amount of administered NK cells is an amount sufficient to reduce the severity of AEs by up to about 1, 2, 3, 4, or 5 Grades when an ICI is administered at about a 10% excess (compared to an amount that is the current standard of care, in an absence of NK cells).

49. The method of any of the preceding arrangements, wherein the effective amount of administered NK cells is an amount sufficient to reduce the severity of AEs by up to about 1, 2, 3, 4, or 5 Grades when an ICI is administered at about a 20% excess (compared to an amount that is the current standard of care, in an absence of NK cells).

50. The method of any of the preceding arrangements, wherein the effective amount of administered NK cells is an amount sufficient to reduce the severity of AEs by up to about 1, 2, 3, 4, or 5 Grades when an ICI is administered at about a 30% excess (compared to an amount that is the current standard of care, in an absence of NK cells).

51. The method of any of the preceding arrangements, wherein the effective amount of administered NK cells is an amount sufficient to reduce the severity of AEs by up to about 1, 2, 3, 4, or 5 Grades when an ICI is administered at about a 40% excess (compared to an amount that is the current standard of care, in an absence of NK cells).

52. The method of any of the preceding arrangements, wherein the effective amount of administered NK cells is an amount sufficient to reduce the severity of AEs by up to about 1, 2, 3, 4, or 5 Grades when an ICI is administered at about a 50% excess (compared to an amount that is the current standard of care, in an absence of NK cells).

53. The method of any of the preceding arrangements, wherein the effective amount of administered NK cells is an amount sufficient to reduce the severity of AEs by up to about 1, 2, 3, 4, or 5 Grades when an ICI is administered at greater than about a 50% excess (compared to an amount that is the current standard of care, in an absence of NK cells).

### Numbered Arrangements 2:

Some embodiments provided herein are described by way of the following provided numbered arrangements and also provided as possible combinations or overlapping embodiments:

1. A method of reducing the likelihood of an adverse event (AE) in a subject treated with an immune checkpoint inhibitor (ICI); the method comprising:
administering an ICI to a subject, and administering a collection of natural killer (NK) cells to the subject; wherein
   NK cells are administered in an amount sufficient to reduce the likelihood and/or severity of one or more AE in the subject as compared to the likelihood and/or severity of the AE in the subject prior to administration of the NK cells.

2. A method of reducing the likelihood of an adverse event (AE) in a subject treated with an anti-PD1 or anti-PDL1 therapy; the method comprising:
administering an anti-PD1 or anti-PDL1 therapy to a subject, and
administering a collection of natural killer (NK) cells to the subject; wherein
   NK cells are administered in an amount sufficient to reduce the likelihood and/or severity of one or more AE in the subject as compared to the likelihood and/or severity of the AE in the subject prior to administration of the NK cells.

3. A method of reducing the likelihood of an adverse event (AE) in a subject treated with pembrolizumab; the method comprising:
administering pembrolizumab to a subject, and
administering CD3−/CD56+ natural killer (NK) cells derived from peripheral blood mononuclear cells (PBMCs) to the subject; wherein
   the NK cells are administered in an amount sufficient to reduce the likelihood and/or severity of one or more AE in the subject as compared to the likelihood and/or severity of the AE in the subject prior to administration of the NK cells.

4. A method of reducing the likelihood of an adverse event (AE) in a subject treated with pembrolizumab; the method comprising:
intravenously administering 200 mg pembrolizumab to a subject, and
administering 4×10⁹ CD3−/CD56+ natural killer (NK) cells derived from peripheral blood mononuclear cells (PBMCs) to the subject; wherein
   administration of the NK cells reduces the likelihood and/or severity of one or more AE in the subject as compared to the likelihood and/or severity of the AE in the subject prior to administration of the NK cells.

5. The method of any of the preceding arrangements, wherein the ICI comprises a PD-1 or PDL1 inhibitor.

6. The method of any of the preceding arrangements, wherein the ICI is an ICI selected from the list comprising: pembrolizumab, nivolumab, atezolizomab, durvalumab, avelumab, camrelizumab, cemiplimab, sintilimab, tisleilizumab, toripalimab, lpilimumab, lpilimumab plus nivolumab, or tremelimumab.

7. The method of any of the preceding arrangements, wherein the ICI is pembrolizumab.

8. The method of any one of the preceding arrangements, wherein between about 1×10⁹−9×10⁹ CD3−/CD56+ natural killer (NK) cells derived from peripheral blood mononuclear cells (PBMCs) are administered to the subject.

9. The method of any one of the preceding arrangements, wherein administration of the NK cells decreases the frequency of diarrhea, colitis, pneumonitis, skin adverse event, endocrine dysfunction, hepatitis, myocarditis, neurotoxicity, renal adverse events, irAEs, and/or any combination thereof, by up to 100% as compared to the frequency of diarrhea, colitis, pneumonitis, skin adverse event, endocrine dysfunction, hepatitis, myocarditis, neurotoxicity, renal adverse events, irAEs, and/or any combination thereof, prior to administration of the NK cells.

10. The method of any one of the preceding arrangements, wherein administration of about 4×10⁹ CD3−/CD56+ natural killer (NK) cells derived from peripheral blood mononuclear cells (PBMCs) decreases the frequency of diarrhea, colitis, pneumonitis, skin adverse event, endocrine dysfunction, hepatitis, myocarditis, neurotoxicity, renal adverse events, irAEs, and/or any combination thereof, in a subject treated with 200 mg pembrolizumab by up to 100% as compared to the frequency of diarrhea, colitis, pneumonitis, skin adverse event, endocrine dysfunction, hepatitis, myocarditis, neurotoxicity, renal adverse events, irAEs, and/or any combination thereof, prior to administration of the SNK01 NK cells.

11. The method of any one of the preceding arrangements, wherein between about 1×10⁹−9×10⁹ CD3−/CD56+ NK cells are administered to the subject.

12. The method of any one of the preceding arrangements, wherein administration of the NK cells decreases the frequency of diarrhea, colitis, pneumonitis, skin adverse event, endocrine dysfunction, hepatitis, myocarditis, neurotoxicity, renal adverse events, irAEs, and/or any combination thereof, by up to 100% as compared to the frequency of diarrhea, colitis, pneumonitis, skin adverse event, endocrine dysfunction, hepatitis, myocarditis, neurotoxicity, renal adverse events, irAEs, and/or any combination thereof, prior to administration of the NK cells.

13. The method of any one of the preceding arrangements, wherein administration of about 4×10⁹ CD3−/CD56+ NK cells decreases the frequency of diarrhea, colitis, pneumonitis, skin adverse event, endocrine dysfunction, hepatitis, myocarditis, neurotoxicity, renal adverse events, irAEs, and/or any combination thereof, in a subject treated with 200 mg pembrolizumab by up to 100% as compared to the frequency of diarrhea, colitis, pneumonitis, skin adverse event, endocrine dysfunction, hepatitis, myocarditis, neurotoxicity, renal adverse events, irAEs, and/or any combination thereof, prior to administration of the CD3−/CD56+NK cells.

14. The method of any one of the preceding arrangements, wherein the ICI and NK cells are administered at the same time.

15. The method of any one of the preceding arrangements, wherein the ICI is administered prior to the NK cells.

16. The method of any one of the preceding arrangements, wherein the NK cells are administered prior to the ICI.

### EXAMPLES

### Production of CD56+ Natural Killer (NK) Cells

CD56+ cells and CD3−/CD56+ cells were isolated from PBMCs by the following method. First, the PBMCs were isolated from the blood using a Ficoll-Hypaque density gradient method and then the cells were counted.

The counted PBMCs were added with a MACS buffer (1×PBS+0.5% HSA) and suspended, and added with CD56 microbeads (Miltenyi Biotec) to be 1 to 20 μL per 1.0×10⁷ PBMCs, and then incubated at 2 to 8° C. for 5 to 30 minutes. After incubation, the MACS buffer was added and mixed, and then the mixture was centrifuged (600×g) to precipitate the cells. After centrifugation, a supernatant was removed, and the cells were suspended by adding the MACS buffer and added in a column connected to a MACS separator. The MACS buffer passed through the column to remove non-specific binding. The column was separated from the MACS separator and transferred to a 15 mL conical tube, and then added with the MACS buffer to isolate CD56+ cells attached to the column.

The counted PBMCs were added with a MACS buffer (1×PBS±0.5% HSA) and suspended, and added with CD3 microbeads (Miltenyi Biotec) to be 1 to 20 μL per 1.0×10⁷ PBMCs, and then incubated at 2 to 8° C. for 5 to 30 minutes. After incubation, the MACS buffer was added and mixed, and then the mixture was centrifuged (600×g) to precipitate the cells. After centrifugation, a supernatant was removed, and the cells were suspended by adding the MACS buffer and added in a column connected to a MACS separator. The MACS buffer passed through the column to collect CD3− cells. The collected CD3− cells were added with a MACS buffer (1×PBS+0.5% HSA) and suspended, and added with CD56 microbeads (Miltenyi Biotec) to be 1 to 20 μL per 1.0×107 CD3− cells, and then incubated at 2 to 8° C. for 5 to 30 minutes. After incubation, the MACS buffer was added and mixed, and then the mixture was centrifuged (600×g) to precipitate the cells. After centrifugation, a supernatant was removed, and the cells were suspended by adding the MACS buffer and added in a column connected to a MACS separator. The MACS buffer passed through the column to remove non-specific binding. The column was separated from the MACS separator and transferred to a 15 mL conical tube, and then added with the MACS buffer to isolate CD3−/CD56+ cells attached to the column.

CD56+ cells or the CD3−/CD56+ cells isolated from PBMCs were added in a RPMI-1640 medium containing FBS 10% added with IL-2 at a concentration of 500 IU/mL together with prepared combination of feeder cells (Jurkat cells and EBV-LCL cells) irradiated with 100 Gy radiation and then co-cultured in an incubator at 37° C. and 5% CO2. The ratio of (CD56+ cells and/or CD3−/CD56+ cells):(Jurkat cells):(EBV-LCL cells) was about 1:30:30.

Meanwhile, the Jurkat cells may be obtained from ATCC (ATCC TIB-152), and the EBV-LCL cells were prepared by the following method: 30×10⁶ PBMCs were added in 9 mL of a culture medium, the mixture was added in a T 25 culture flask, and then 9 m of an EBV supernatant was added. 80 μL of cyclosporine A was added and then cultured at 37° C. After 7 days of culture, a half of supernatant was removed, a fresh culture medium was added, and then 40 μL of cyclosporine A was added. The same process as the 7th day was repeated once every 7 days until 28 days of culture. The cell line was usable after 28 days of culture, and from this time, the cell line was cultured in the culture medium without adding cyclosporine A.

### Production of CD56+CD3− Natural Killer (NK) Cells (IL-2/IL-21 Treated)

CD3−/CD56+ cells were isolated from PBMCs by the following method. First, the PBMCs were isolated from the blood using a Ficoll-Hypaque density gradient method and then the cells were counted.

The counted PBMCs were added with a MACS buffer (1×PBS+0.5% HSA) and suspended, and added with CD56 microbeads (Miltenyi Biotec) to be 1 to 20 μL per 1.0×10⁷ PBMCs, and then incubated at 2 to 8° C. for 5 to 30 minutes. After incubation, the MACS buffer was added and mixed, and then the mixture was centrifuged (600×g) to precipitate the cells. After centrifugation, a supernatant was removed, and the cells were suspended by adding the MACS buffer and added in a column connected to a MACS separator. The MACS buffer passed through the column to remove non-specific binding. The column was separated from the MACS separator and transferred to a 15 mL conical tube, and then added with the MACS buffer to isolate CD56+ cells attached to the column.

The counted PBMCs were added with a MACS buffer (1×PBS±0.5% HSA) and suspended, and added with CD3 microbeads (Miltenyi Biotec) to be 1 to 20 μL per 1.0×10⁷ PBMCs, and then incubated at 2 to 8° C. for 5 to 30 minutes. After incubation, the MACS buffer was added and mixed, and then the mixture was centrifuged (600×g) to precipitate the cells. After centrifugation, a supernatant was removed, and the cells were suspended by adding the MACS buffer and added in a column connected to a MACS separator. The MACS buffer passed through the column to collect CD3− cells. The collected CD3− cells were added with a MACS buffer (1×PBS+0.5% HSA) and suspended, and added with CD56 microbeads (Miltenyi Biotec) to be 1 to 20 μL per 1.0×10⁷ CD3− cells, and then incubated at 2 to 8° C. for 5 to 30 minutes. After incubation, the MACS buffer was added and mixed, and then the mixture was centrifuged (600×g) to precipitate the cells. After centrifugation, a supernatant was removed, and the cells were suspended by adding the MACS buffer and added in a column connected to a MACS separator. The MACS buffer passed through the column to remove non-specific binding. The column was separated from the MACS separator and transferred to a 15 mL conical tube, and then added with the MACS buffer to isolate CD3−/CD56+ cells attached to the column.

CD56+ cells or the CD3−/CD56+ cells isolated from PBMCs were added in a RPMI-1640 medium containing FBS 10% added with IL-2 (500 IU/mL) and IL-21 (50 ng/mL) together with prepared combination of feeder cells (Jurkat cells and EBV-LCL cells) irradiated with 100 Gy radiation and then co-cultured in an incubator at 37° C. and 5% CO2. The ratio of (CD56+ cells and/or CD3−/CD56+ cells):(Jurkat cells):(EBV-LCL cells) was about 1:30:30.

Meanwhile, the Jurkat cells may be obtained from ATCC (ATCC TIB-152), and the EBV-LCL cells were prepared by the following method: 30×10⁶ PBMCs were added in 9 mL of a culture medium, the mixture was added in a T 25 culture flask, and then 9 m of an EBV supernatant was added. 80 μL of cyclosporine A was added and then cultured at 37° C. After 7 days of culture, a half of supernatant was removed, a fresh culture medium was added, and then 40 μL of cyclosporine A was added. The same process as the 7th day was repeated once every 7 days until 28 days of culture. The cell line was usable after 28 days of culture, and from this time, the cell line was cultured in the culture medium without adding cyclosporine A.

Production of Natural Killer (NK) Cells without the CD56+ Cells Isolation Step (IL-2 Treated)

PBMCs were isolated from the blood using a Ficoll-Hypaque density gradient method. The PBMCs were added in a RPMI-1640 medium containing FBS 10% added with IL-2 at a concentration of 500 IU/mL together with prepared feeder cells (Jurkat cells and EBV-LCL cells) irradiated with 100 Gy radiation and then co-cultured in an incubator at 37° C. and 5% CO2.

Production of Natural Killer (NK) Cells without the CD56+ Cells Isolation Step (IL-2/IL-21 Treated)

PBMCs were isolated from the blood using a Ficoll-Hypaque density gradient method. The PBMCs were added in a RPMI-1640 medium containing FBS 10% added with IL-2 (500 IU/mL) and IL-21 (50 ng/mL) together with prepared feeder cells (Jurkat cells and EBV-LCL cells) irradiated with 100 Gy radiation and then co-cultured in an incubator at 37° C. and 5% CO2.

Production of Natural Killer (NK) Cells without the CD56+ Cells Isolation Step

PBMCs were isolated from the blood using a Ficoll-Hypaque density gradient method. The PBMCs were added in a RPMI-1640 medium containing FBS 10% added with IL-2 (500 IU/mL) and IL-21 (50 ng/mL) together with prepared feeder cells (Jurkat cells and EBV-LCL cells at a ratio of 1:0.5:0.5) irradiated with 100 Gy radiation and then co-cultured in an incubator at 37° C. and 5% CO2.

### Avelumab in Metastatic or Locally Advanced Solid Tumors (JAVELIN Solid Tumor)

A Phase 1, open-label, dose-escalation trial of avelumab [antibody targeting programmed death ligand 1 (anti PD-L1)] with consecutive parallel group expansion in 1783 participants with selected tumor indications was performed. The cohorts comprised non-small cell lung cancer (NSCLC, first line), NSCLC (post-platinum), metastatic breast cancer (MBC), colorectal cancer (CRC), urothelial carcinoma (secondary), mesothelioma, gastric/GEJ cancer (first line switch maintenance and second line), and ovarian cancer (secondary and platinum refractory+liposomal doxorubicin), renal cell carcinoma (second line) melanoma and head, neck squamous cell carcinoma (HNSCC), castrate-resistant prostate cancer (CRPC), adrenocortical carcinoma (ACC) urothelial carcinoma (efficacy), gastric/gastroesophageal junction (GEJ) cancer (third line), renal cell carcinoma (RCC, first line) and escalation phase.

Safety data from 1783 patients enrolled in the Javelin Solid Tumor and Merkel 200 trials with avelumab monotherapy revealed 16.5% of patients experienced irAEs and 25.5% had infusion related reactions. 10% experienced Grade 3 diarrhea and 13% had Grade 3 rash.

Tables 2-5 show a summary of the safety results of the study. Table 2 shows the frequency of treatment emergent adverse events (TEAEs) in the dose escalation cohort.

TABLE 2

| | | | | | Dose |
|---|---|---|---|---|---|
| | | | Dose | Dose | Escalation |
| | Dose | Dose | Escalation | Escalation | Cohort: |
| | Escalation | Escalation | Cohort: | Cohort: | Avelumab |
| | Cohort: | Cohort: | Avelumab | Avelumab | 10.0 |
| | Avelumab | Avelumab | 10.0 | 20.0 | mg/kg |
| | 1.0 mg/kg | 3.0 mg/kg | mg/kg | mg/kg | Weekly |
| Overall | 4 | 13 | 15 | 21 | 8 |
| Number of | | | | | |
| Participants | | | | | |
| Analyzed | | | | | |
| Participants | 4 | 13 | 15 | 21 | 8 |
| with TEAEs | | | | | |
| Participants | 0 | 1 | 1 | 2 | 2 |
| with TEAEs | | | | | |
| with Grade 1 | | | | | |
| severity | | | | | |
| Participants | 0 | 4 | 4 | 6 | 3 |
| with TEAEs | | | | | |
| with Grade 2 | | | | | |
| severity | | | | | |
| Participants | 2 | 4 | 6 | 11 | 3 |
| with TEAEs | | | | | |
| with Grade 3 | | | | | |
| severity | | | | | |
| Participants | 0 | 0 | 2 | 1 | 0 |
| with TEAEs | | | | | |
| with Grade 4 | | | | | |
| severity | | | | | |
| Participants | 2 | 4 | 2 | 1 | 0 |
| with TEAEs | | | | | |
| with Grade 5 | | | | | |
| severity | | | | | |
| Participants | 0 | 0 | 0 | 0 | 0 |
| with TEAEs | | | | | |
| with missing | | | | | |
| Grade | | | | | |

Experimental: Dose Escalation Cohort: Avelumab 1.0 mg/kg. Participants with metastatic or locally advanced solid tumors received intravenous infusion of Avelumab at a dose of 1.0 milligrams per kilogram (mg/kg) once every 2 weeks in dose escalation cohort until confirmed progression, unacceptable toxicity, or any reason for withdrawal from the trial or investigational medicinal product (IMP) occurs.

Experimental: Dose Escalation Cohort: Avelumab 3.0 mg/kg

Participants with metastatic or locally advanced solid tumors received intravenous infusion of Avelumab at a dose of 3.0 mg/kg once every 2 weeks in dose escalation cohort until confirmed progression, unacceptable toxicity, or any reason for withdrawal from the trial or IP occurs.

Experimental: Dose Escalation Cohort: Avelumab 20.0 mg/kg

Participants with metastatic or locally advanced solid tumors received intravenous infusion of Avelumab at a dose of 20.0 mg/kg once every 2 weeks in dose escalation cohort until confirmed progression, unacceptable toxicity, or any reason for withdrawal from the trial or IMP occurs.

Experimental: Dose Escalation Cohort: Avelumab 10.0 mg/kg Weekly

Participants with metastatic or locally advanced solid tumors received intravenous infusion of Avelumab at a dose of 10.0 mg/kg once weekly for the first 12 weeks and once every 2 weeks starting Week 13 in dose escalation cohort until confirmed progression, unacceptable toxicity, or any reason for withdrawal from the trial or IMP occurs.

Table 3 shows the frequency of TEAEs in the primary expansion cohort.

TABLE 3

| | Primary | Primary | Primary | | |
|---|---|---|---|---|---|
| | Expansion | Expansion | Expansion | Primary | Primary |
| | Cohort: NSCLC, | Cohort: | Cohort: | Expansion | Expansion |
| | Post-platinum | NSCLC, | Metastatic Breast | Cohort: GC/GEJC | Cohort: GC/GEJC |
| | Doublet | First Line | Cancer | Progressed | Non Progressed |
| Overall | 184 | 156 | 168 | 60 | 90 |
| Number of | | | | | |
| Participants | | | | | |
| Analyzed | | | | | |
| Participants | 182 | 156 | 161 | 60 | 88 |
| with TEAEs | | | | | |
| Participants | 11 | 3 | 22 | 5 | 14 |
| with TEAEs | | | | | |
| with Grade | | | | | |
| 1 severity | | | | | |
| Participants | 54 | 45 | 58 | 9 | 19 |
| with TEAEs | | | | | |
| with Grade | | | | | |
| 2 severity | | | | | |
| Participants | 66 | 68 | 45 | 29 | 41 |
| with TEAEs | | | | | |
| with Grade | | | | | |
| 3 severity | | | | | |
| Participants | 19 | 15 | 12 | 7 | 4 |
| with TEAEs | | | | | |
| with Grade | | | | | |
| 4 severity | | | | | |
| Participants | 32 | 25 | 24 | 10 | 10 |
| with TEAEs | | | | | |
| with Grade | | | | | |
| 5 severity | | | | | |
| Participants | 0 | 0 | 0 | 0 | 0 |
| with TEAEs | | | | | |
| with | | | | | |
| missing | | | | | |
| Grade | | | | | |

### Experimental: Primary Expansion Cohort: NSCLC, Post-Platinum Doublet

Participants with non-small cell lung cancer (NSCLC), who had progressed after 1 line of platinum-containing doublet chemotherapy received intravenous infusion of Avelumab at a dose of 10.0 mg/kg once every 2 weeks in primary expansion cohort until confirmed progression, unacceptable toxicity, or any reason for withdrawal from the trial or IMP occurs.

### Experimental: Primary Expansion Cohort: NSCLC, First Line

Participants with non-small cell lung cancer (NSCLC), first line received intravenous infusion of Avelumab at a dose of 10.0 mg/kg once every 2 weeks in primary expansion cohort until confirmed progression, unacceptable toxicity, or any reason for withdrawal from the trial or IMP occurs.

### Experimental: Primary Expansion Cohort: Metastatic Breast Cancer

Participants with metastatic breast cancer received intravenous infusion of Avelumab at a dose of 10.0 mg/kg once every 2 weeks in primary expansion cohort until confirmed progression, unacceptable toxicity, or any reason for withdrawal from the trial or IMP occurs.

### Experimental: Primary Expansion Cohort: GC/GEJC Progressed

Participants with gastric (GC) and gastroesophageal junction cancer (GEJC) who progressed on or after first line chemotherapy received intravenous infusion of Avelumab at a dose of 10.0 mg/kg once every 2 weeks in primary expansion cohort until confirmed progression, unacceptable toxicity, or any reason for withdrawal from the trial or IMP occurs.

### Experimental: Primary Expansion Cohort: GC/GEJC Non Progressed

Participants with gastric (GC) and gastroesophageal junction cancer (GEJC) who non-progressed on or after first-line chemotherapy received intravenous infusion of Avelumab at a dose of 10.0 mg/kg once every 2 weeks in primary expansion cohort until confirmed progression, unacceptable toxicity, or any reason for withdrawal from the trial or IMP occurs.

Table 4 shows the frequency of TEAEs in the secondary expansion cohort.

TABLE 4

| | | | | | | | | Secondary | Secondary |
|---|---|---|---|---|---|---|---|---|---|
| | | Secondary | | | | | | Expansion | Expansion |
| | | Expansion | | | | | | Cohort: | Cohort: |
| | Secondary | Cohort: | Secondary | | | Secondary | Secondary | Renal | Renal |
| | Expansion | Castrate- | Expansion | Secondary | Secondary | Expansion | Expansion | Cell | Cell |
| | Cohort: | resistant | Cohort: | Expansion | Expansion | Cohort: | Cohort: | Carcinoma | Carcinoma |
| | Colorectal | Prostate | Adrenocortical | Cohort: | Cohort: | Urothelial | Ovarian | (First | (Second |
| | Cancer | Cancer | Carcinoma | Melanoma | Mesothelioma | Carcinoma | Cancer | Line) | Line) |
| Overall | 21 | 18 | 50 | 51 | 53 | 44 | 125 | 62 | 20 |
| Number | | | | | | | | | |
| of | | | | | | | | | |
| Participants | | | | | | | | | |
| Analyzed | | | | | | | | | |
| Participants | 21 | 17 | 50 | 50 | 53 | 44 | 122 | 62 | 19 |
| with | | | | | | | | | |
| TEAEs | | | | | | | | | |
| Participants | 2 | 5 | 1 | 2 | 2 | 3 | 11 | 7 | 1 |
| with | | | | | | | | | |
| TEAEs | | | | | | | | | |
| with | | | | | | | | | |
| Grade 1 | | | | | | | | | |
| severity | | | | | | | | | |
| Participants | 9 | 5 | 10 | 17 | 21 | 10 | 52 | 25 | 5 |
| with | | | | | | | | | |
| TEAEs | | | | | | | | | |
| with | | | | | | | | | |
| Grade 2 | | | | | | | | | |
| severity | | | | | | | | | |
| Participants | 6 | 7 | 25 | 21 | 23 | 18 | 42 | 20 | 10 |
| with | | | | | | | | | |
| TEAEs | | | | | | | | | |
| with | | | | | | | | | |
| Grade 3 | | | | | | | | | |
| severity | | | | | | | | | |
| Participants | 1 | 0 | 8 | 5 | 3 | 6 | 3 | 6 | 1 |
| with | | | | | | | | | |
| TEAEs | | | | | | | | | |
| with | | | | | | | | | |
| Grade 4 | | | | | | | | | |
| severity | | | | | | | | | |
| Participants | 3 | 0 | 6 | 5 | 4 | 7 | 14 | 4 | 2 |
| with | | | | | | | | | |
| TEAEs | | | | | | | | | |
| with | | | | | | | | | |
| Grade 5 | | | | | | | | | |
| severity | | | | | | | | | |
| Participants | 0 | 0 | 0 | 0 | 0 | 0 | 0 | 0 | 0 |
| with | | | | | | | | | |
| TEAEs | | | | | | | | | |
| with | | | | | | | | | |
| missing | | | | | | | | | |
| Grade | | | | | | | | | |

### Experimental: Secondary Expansion Cohort: Colorectal Cancer

Participants with colorectal cancer received intravenous infusion of Avelumab at a dose of 10.0 mg/kg once every 2 weeks in secondary expansion cohort until confirmed progression, unacceptable toxicity, or any reason for withdrawal from the trial or IMP occurs.

### Experimental: Secondary Expansion Cohort: Castrate-Resistant Prostate Cancer

Participants with castrate-resistant prostate cancer received intravenous infusion of Avelumab at a dose of 10.0 mg/kg once every 2 weeks in secondary expansion cohort until confirmed progression, unacceptable toxicity, or any reason for withdrawal from the trial or IMP occurs.

### Experimental: Secondary Expansion Cohort: Adrenocortical Carcinoma

Participants with adrenocortical carcinoma received intravenous infusion of Avelumab at a dose of 10.0 mg/kg once every 2 weeks in secondary expansion cohort until confirmed progression, unacceptable toxicity, or any reason for withdrawal from the trial or IMP occurs.

### Experimental: Secondary Expansion Cohort: Melanoma

Participants with melanoma received intravenous infusion of Avelumab at a dose of 10.0 mg/kg once every 2 weeks in secondary expansion cohort until confirmed progression, unacceptable toxicity, or any reason for withdrawal from the trial or IMP occurs.

### Experimental: Secondary Expansion Cohort: Mesothelioma

Participants with mesothelioma received intravenous infusion of Avelumab at a dose of 10.0 mg/kg once every 2 weeks in secondary expansion cohort until confirmed progression, unacceptable toxicity, or any reason for withdrawal from the trial or IMP occurs.

### Experimental: Secondary Expansion Cohort: Urothelial Carcinoma

Participants with urothelial carcinoma received intravenous infusion of Avelumab at a dose of 10.0 mg/kg once every 2 weeks in secondary expansion cohort until confirmed progression, unacceptable toxicity, or any reason for withdrawal from the trial or IMP occurs.

### Experimental: Secondary Expansion Cohort: Ovarian Cancer

Participants with ovarian carcinoma received intravenous infusion of Avelumab at a dose of 10.0 mg/kg once every 2 weeks in secondary expansion cohort until confirmed progression, unacceptable toxicity, or any reason for withdrawal from the trial or IMP occurs.

### Experimental: Secondary Expansion Cohort: Renal Cell Carcinoma (First Line)

Participants with Renal cell carcinoma received intravenous infusion of Avelumab at a dose of 10.0 mg/kg once every 2 weeks as a first-line therapy in secondary expansion cohort until confirmed progression, unacceptable toxicity, or any reason for withdrawal from the trial or IMP occurs.

### Experimental: Secondary Expansion Cohort: Renal Cell Carcinoma (Second Line)

Participants with Renal cell carcinoma who failed 1 prior systemic first-line regimen received intravenous infusion of Avelumab at a dose of 10.0 mg/kg once every 2 weeks as a second line treatment in secondary expansion cohort until confirmed progression, unacceptable toxicity, or any reason for withdrawal from the trial or IMP occurs.

Table 5 shows the frequency of TEAEs in the efficacy expansion cohort.

TABLE 5

| | Efficacy | Efficacy | Efficacy | |
|---|---|---|---|---|
| | Expansion | Expansion | Expansion | Efficacy |
| | Cohort: | Cohort: | Cohort: | Expansion |
| | Ovarian | Urothelial | GC/GEJC, | Cohort: |
| | Cancer | Carcinoma | Third Line | HNSCC |
| Overall | 103 | 205 | 132 | 153 |
| Number of | | | | |
| Participants | | | | |
| Analyzed | | | | |
| Participants | 103 | 204 | 130 | 149 |
| with TEAEs | | | | |
| Participants | 7 | 15 | 2 | 14 |
| with TEAEs | | | | |
| with Grade | | | | |
| 1 severity | | | | |
| Participants | 30 | 46 | 30 | 43 |
| with TEAEs | | | | |
| with Grade | | | | |
| 2 severity | | | | |
| Participants | 45 | 82 | 51 | 54 |
| with TEAEs | | | | |
| with Grade | | | | |
| 3 severity | | | | |
| Participants | 6 | 16 | 12 | 12 |
| with TEAEs | | | | |
| with Grade | | | | |
| 4 severity | | | | |
| Participants | 15 | 45 | 35 | 25 |
| with TEAEs | | | | |
| with Grade | | | | |
| 5 severity | | | | |
| Participants | 0 | 0 | 0 | 1 |
| with TEAEs | | | | |
| with | | | | |
| missing | | | | |
| Grade | | | | |

### Experimental: Efficacy Expansion Cohort: Ovarian Cancer

Participants with ovarian carcinoma received intravenous infusion of Avelumab at a dose of 10.0 mg/kg once every 2 weeks in efficacy expansion cohort until confirmed progression, unacceptable toxicity, or any reason for withdrawal from the trial or IMP occurs.

### Experimental: Efficacy Expansion Cohort: Urothelial Carcinoma

Participants with urothelial carcinoma received intravenous infusion of Avelumab at a dose of 10.0 mg/kg once every 2 weeks in efficacy expansion cohort until confirmed progression, unacceptable toxicity, or any reason for withdrawal from the trial or IMP occurs.

### Experimental: Efficacy Expansion Cohort: GC/GEJC, Third Line

Participants with gastric (GC) and gastroesophageal junction cancer (GEJC) who have failed both a first-line chemotherapy regimen and subsequent ramucirumab therapy, received intravenous infusion of Avelumab at a dose of 10.0 mg/kg once every 2 weeks as a third-line treatment in efficacy expansion cohort until confirmed progression, unacceptable toxicity, or any reason for withdrawal from the trial or IMP occurs.

### Experimental: Efficacy Expansion Cohort: HNSCC

Participants with head and neck squamous cell carcinoma (HNSCC) received intravenous infusion of Avelumab at a dose of 10.0 mg/kg once every 2 weeks in efficacy expansion cohort until confirmed progression, unacceptable toxicity, or any reason for withdrawal from the trial or IMP occurs.

### Primary Outcome Measures:

Dose Escalation Cohort (Excluding Once Weekly Avelumab 10 mg/kg Cohort): Number of Participants Experiencing Dose Limiting Toxicities (DLTs) [Time Frame: Dose Escalation: Baseline up to Week 3]

DLT: defined using National Cancer Institute Common Toxicity Criteria for Adverse Events Version 4.0, as any one of following: any Grade (Gr) >=3toxicity that is possibly/probably/definitely related to avelumab, except for any of following: Gr 3 infusion-related reaction resolving within 6 hours and controlled with medical management, Transient Gr 3 flu-like symptoms/fever, which is controlled with medical management, Transient Gr 3 fatigue, local reactions, headache, nausea, emesis that resolves to <=Gr 1, Gr3 diarrhea, Gr 3 skin toxicity, Gr 3 liver function test increase that resolves to <=Gr1 in <7 days after medical management has been initiated, Single laboratory values out of normal range that were unlikely related to study treatment according to investigator, did not have any clinical correlate, and resolved to <=Gr1 within 7 days with adequate medical management and tumor flare phenomenon defined as local pain, irritation/rash localized at sites of known/suspected tumor.

Efficacy Expansion Cohort (Ovarian Cancer): Number of Participants With Confirmed Best Overall Response (BOR) as Per Response Evaluation Criteria in Solid Tumors Version 1.1 (RECIST 1.1), as Adjudicated by Independent Endpoint Review Committee (IERC) [Time Frame: Ovarian Cancer Efficacy Expansion: Baseline up to Day 620]

Confirmed BOR was determined according to RECIST 1.1 and as adjudicated by an Independent Endpoint Review Committee (IERC) and defined as best response of any of complete response (CR), partial response (PR), stable disease (SD) and progressive disease (PD) recorded from date of randomization until disease progression/recurrence (taking smallest measurement recorded since start of treatment as reference). CR: Disappearance of all evidence of target/non-target lesions. PR: At least 30% reduction from baseline in sum of longest diameter (SLD) of all lesions. SD: Neither sufficient increase to qualify for PD nor sufficient shrinkage to qualify for PR. PD: at least a 20% increase in SLD, taking as reference smallest SLD recorded from baseline/appearance of 1 or more new lesions and unequivocal progression of non-target lesions.

Efficacy Expansion Cohort (Urothelial Carcinoma): Number of Participants With Confirmed Best Overall Response (BOR) as Per Response Evaluation Criteria in Solid Tumors Version 1.1 (RECIST 1.1), as Adjudicated by Independent Endpoint Review Committee (IERC) [Time Frame: Urothelial Carcinoma Efficacy Expansion: Baseline up to Day 931].

Confirmed BOR was determined according to RECIST 1.1 and as adjudicated by an Independent Endpoint Review Committee (IERC) and defined as best response of any of complete response (CR), partial response (PR), stable disease (SD) and progressive disease (PD) recorded from date of randomization until disease progression/recurrence (taking smallest measurement recorded since start of treatment as reference). CR: Disappearance of all evidence of target/non-target lesions. PR: At least 30% reduction from baseline in sum of longest diameter (SLD) of all lesions. SD: Neither sufficient increase to qualify for PD nor sufficient shrinkage to qualify for PR. PD: at least a 20% increase in SLD, taking as reference smallest SLD recorded from baseline/appearance of 1 or more new lesions and unequivocal progression of non-target lesions.

Efficacy Expansion Cohort (GC/GEJC, Third Line): Number of Participants With Confirmed Best Overall Response (BOR) as Per Response Evaluation Criteria in Solid Tumors Version 1.1 (RECIST 1.1), as Adjudicated by Independent Endpoint Review Committee (IERC) [Time Frame: GC/GEJC, Third Line Efficacy Expansion: Baseline up to Day 871].

Confirmed BOR was determined according to RECIST 1.1 and as adjudicated by an Independent Endpoint Review Committee (IERC) and defined as best response of any of complete response (CR), partial response (PR), stable disease (SD) and progressive disease (PD) recorded from date of randomization until disease progression/recurrence (taking smallest measurement recorded since start of treatment as reference). CR: Disappearance of all evidence of target/non-target lesions. PR: At least 30% reduction from baseline in sum of longest diameter (SLD) of all lesions. SD: Neither sufficient increase to qualify for PD nor sufficient shrinkage to qualify for PR. PD: at least a 20% increase in SLD, taking as reference smallest SLD recorded from baseline/appearance of 1 or more new lesions and unequivocal progression of non-target lesions.

Efficacy Expansion Cohort (HNSCC): Number of Participants With Confirmed Best Overall Response (BOR) as Per Response Evaluation Criteria in Solid Tumors Version 1.1 (RECIST 1.1), as Adjudicated by Independent Endpoint Review Committee (IERC) [Time Frame: HNSCC Efficacy Expansion: Baseline up to Day 1072]

Confirmed BOR was determined according to RECIST 1.1 and as adjudicated by an Independent Endpoint Review Committee (IERC) and defined as best response of any of complete response (CR), partial response (PR), stable disease (SD) and progressive disease (PD) recorded from date of randomization until disease progression/recurrence (taking smallest measurement recorded since start of treatment as reference). CR: Disappearance of all evidence of target/non-target lesions. PR: At least 30% reduction from baseline in sum of longest diameter (SLD) of all lesions. SD: Neither sufficient increase to qualify for PD nor sufficient shrinkage to qualify for PR. PD: at least a 20% increase in SLD, taking as reference smallest SLD recorded from baseline/appearance of 1 or more new lesions and unequivocal progression of non-target lesions.

### Secondary Outcome Measures:

Dose Escalation and Expansion Cohorts: Number of Participants With TEAEs and TEAEs as Per Severity [Time Frame: Up to Day 2511]

Adverse event (AE): any unfavorable and unintended sign (including an abnormal laboratory finding), symptom, or disease temporally associated with use of study drug, whether or not related to study drug. A serious adverse event (SAE) was an AE that resulted in any of following outcomes: death; life threatening; persistent/significant disability/incapacity; initial or prolonged inpatient hospitalization; congenital anomaly/birth defect or was otherwise considered medically important. Treatment-emergent events were events between first dose of study drug that were absent before treatment or that worsened relative to pre-treatment state up to 30 days after last administration. TEAEs included both Serious TEAEs and non-serious TEAEs. Severity of TEAEs were graded using National Cancer Institute-Common Terminology Criteria for Adverse Events (NCI-CTCAE) version 4.0 toxicity grades, as follows: Grade 1=Mild, Grade 2=Moderate, Grade 3=Severe, Grade 4=Life-threatening and Grade 5=Death.

Dose Escalation and Expansion Cohorts: Number of Participants With Treatment-Related Treatment-Emergent Adverse Events (TEAEs) and Treatment-Related TEAEs as Per Severity [Time Frame: Baseline up to Day 2511]

AE was defined as any unfavorable and unintended sign (including an abnormal laboratory finding), symptom, or disease temporally associated with the use of study drug, whether or not related to study drug. Treatment-emergent events were the events between first dose of study drug that were absent before treatment or that worsened relative to pre-treatment state up to 30 days after last administration. TEAEs included both Serious TEAEs and non-serious TEAEs. Treatment related AE was defined as having a “Possible” or “Related” relationship to study treatment, as assessed by the Investigator. Severity of Treatment-Related TEAEs were graded using NCI-CTCAE version 4.0 toxicity grades, as follows: Grade 1=Mild, Grade 2=Moderate, Grade 3=Severe, Grade 4=Life-threatening and Grade 5=Death.

Dose Escalation Cohort (Excluding Once Weekly Avelumab 10 mg/kg Cohort): Area Under Serum Concentration-Time Curve From the Time of Dosing to the Time of the Last Observation (AUC0-t) of Avelumab [Time Frame: Pre-infusion, at end of 1-hour infusion (Day 1), 0.5, 1, 2, 4, 6, 12, 24, 36, 48 hours after end of infusion].

Area under the serum concentration versus time curve from time zero to the last sampling time t at which the concentration is at or above the lower limit of quantification (LLLQ). AUC(0-t) was calculated according to the mixed log-linear trapezoidal rule.

Dose Escalation Cohort (Excluding Once Weekly Avelumab 10 mg/kg Cohort): Area Under the Serum Concentration-Time Curve From Time Zero to Infinity (AUC0-infinity) of Avelumab [Time Frame: Pre-infusion, at end of 1-hour infusion (Day 1), 0.5, 1, 2, 4, 6, 12, 24, 36, 48 hours after end of infusion].

The AUC(0-inf) was estimated by determining the total area under the curve of the concentration versus time curve extrapolated to infinity.

Dose Escalation Cohort (Excluding Once Weekly Avelumab 10 mg/kg Cohort): Maximum Observed Serum Concentration (Cmax) of Avelumab [Time Frame: Pre-infusion, at end of 1-hour infusion (Day 1), 0.5, 1, 2, 4, 6, 12, 24, 36, 48 hours after end of infusion].

Cmax is the maximum observed serum concentration obtained directly from the concentration versus time curve.

Dose Escalation Cohort (Excluding Once Weekly Avelumab 10 mg/kg Cohort): Time to Reach Maximum Observed Serum Concentration (Tmax) of Avelumab [Time Frame: Pre-infusion, at end of 1-hour infusion (Day 1), 0.5, 1, 2, 4, 6, 12, 24, 36, 48 hours after end of infusion].

Tmax is time to reach maximum observed serum concentration obtained directly from the concentration versus time curve.

Dose Escalation Cohort (Excluding Once Weekly Avelumab 10 mg/kg Cohort): Apparent Terminal Half-Life (t½) of Avelumab [Time Frame: Pre-infusion, at end of 1-hour infusion (Day 1), 0.5, 1, 2, 4, 6, 12, 24, 36, 48 hours after end of infusion].

Apparent terminal half-life was defined as the time required for the serum concentration of drug to decrease 50 percent in the final stage of its elimination.

Dose Expansion Phase: Serum Concentration at End of Infusion (CEOI) of Avelumab [Time Frame: At Day 1, 15, 29, 43, 85, 127 and 169].

Serum concentration at end of infusion (CEOI) of Avelumab is reported.

Dose Expansion Phase: Minimum Serum Post-dose (Ctrough) Concentration of Avelumab [Time Frame: At Day 15, 29, 43, 57, 71, 85, 99, 127 and 169].

Serum Ctrough concentration of Avelumab is reported.

Dose Escalation Cohort (Excluding Once Weekly Avelumab 10 mg/kg Cohort): Number of Participants With Immune Related Best Overall Response (irBOR) According to Modified Immune-Related Response Criteria (irRC) [Time Frame: Dose Escalation: Baseline up to Day 1023].

irBOR defined as best response of any of immune related complete response (irCR), immune related partial response (irPR), immune related stable disease (irSD) and immune related progressive disease (irPD) recorded from baseline until immune related disease progression and determined according to modified irRC per investigator assessment. irCR: Complete disappearance of all tumor lesions (both index and non-index lesions with no new measurable/unmeasurable lesions). irPR: At least 30% reduction from baseline in the sum of the longest diameter (SLD) of all lesions). irSD: SLD of target and new measurable lesions neither irCR, irPR, or irPD. irPD: SLD of target and new measurable lesions increases greater than or equal to [>=]20%, confirmed by a repeat, consecutive observations at least 4 weeks from the date first documented. Number of participants with immune-related best overall response in each category (irCR, irPR, irSD, irPD) was reported.

Dose Expansion Cohort: Number of Participants With Immune Related Best Overall Response (irBOR) According to Modified Immune-Related Response Criteria (irRC) [Time Frame: Dose Expansion: Baseline up to Day 2023].

irBOR defined as best response of any of immune related complete response (irCR), immune related partial response (irPR), immune related stable disease (irSD) and immune related progressive disease (irPD) recorded from baseline until immune related disease progression and determined according to modified irRC per investigator assessment. irCR: Complete disappearance of all tumor lesions (both index and non-index lesions with no new measurable/unmeasurable lesions). irPR: At least 30% reduction from baseline in the sum of the longest diameter (SLD) of all lesions). irSD: SLD of target and new measurable lesions neither irCR, irPR, or irPD. irPD: SLD of target and new measurable lesions increases greater than or equal to [>=]20%, confirmed by a repeat, consecutive observations at least 4 weeks from the date first documented. Number of participants with immune-related best overall response in each category (irCR, irPR, irSD, irPD) was reported.

Dose Escalation Cohort: Number of Participants With Best Overall Response (BOR) According to Response Evaluation Criteria in Solid Tumors (RECIST) Version 1.1 [Time Frame: Dose Escalation: Baseline up to Day 2511].

BOR was determined according to RECIST v1.1 and as per investigator assessment. BOR is defined as the best response of any of complete response (CR), partial response (PR), stable disease (SD) and progressive disease (PD) recorded from date of randomization until disease progression or recurrence (taking the smallest measurement recorded since start of treatment as reference). CR: Disappearance of all evidence of target and non-target lesions. PR: At least 30 percent (%) reduction from baseline in the sum of the longest diameter (SLD) of all lesions. SD=Neither sufficient increase to qualify for PD nor sufficient shrinkage to qualify for PR. PD is defined as at least a 20% increase in the SLD, taking as reference the smallest SLD recorded from baseline or appearance of 1 or more new lesions and unequivocal progression of non-target lesions. Number of participants with best overall response in each category (CR, PR, SD, PD) was reported.

Dose Expansion Cohort: Number of Participants With Best Overall Response (BOR) According to Response Evaluation Criteria in Solid Tumors (RECIST) Version 1.1 [Time Frame: Dose Expansion: Baseline up to Day 2023].

BOR was determined according to RECIST v1.1 and as per investigator assessment. BOR is defined as the best response of any of complete response (CR), partial response (PR), stable disease (SD) and progressive disease (PD) recorded from date of randomization until disease progression or recurrence (taking the smallest measurement recorded since start of treatment as reference). CR: Disappearance of all evidence of target and non-target lesions. PR: At least 30 percent (%) reduction from baseline in the sum of the longest diameter (SLD) of all lesions. SD=Neither sufficient increase to qualify for PD nor sufficient shrinkage to qualify for PR. PD is defined as at least a 20% increase in the SLD, taking as reference the smallest SLD recorded from baseline or appearance of 1 or more new lesions and unequivocal progression of non-target lesions. Number of participants with best overall response in each category (CR, PR, SD, PD) was reported.

Dose Expansion Cohort (Secondary Urothelial Carcinoma Cohort): Number of Participants With Confirmed Best Overall Response According to Response Evaluation Criteria in Solid Tumors Version 1.1 as Adjudicated by an Independent Endpoint Review Committee [Time Frame: Secondary Urothelial Carcinoma Dose Expansion: Baseline up to Day 931].

Confirmed Best Overall Response (BOR) was determined according to Response Evaluation Criteria in Solid Tumors (RECIST) v1.1 and as adjudicated by an Independent Endpoint Review Committee (IERC) is defined as best response of any of complete response (CR), partial response (PR), stable disease (SD) and progressive disease (PD) recorded from date of randomization until disease progression/recurrence (taking smallest measurement recorded since start of treatment as reference). CR: Disappearance of all evidence of target/non-target lesions. PR: At least 30% reduction from baseline in sum of longest diameter (SLD) of all lesions. SD: Neither sufficient increase to qualify for PD nor sufficient shrinkage to qualify for PR. PD: at least a 20% increase in SLD, taking as reference smallest SLD recorded from baseline/appearance of 1 or more new lesions and unequivocal progression of non-target lesions. Number of participants with BOR in each category (CR, PR, SD, PD) were reported.

Dose Expansion Cohort: Progression-Free Survival (PFS) Time According to Response Evaluation Criteria in Solid Tumors Version (RECIST) 1.1 [Time Frame: Dose Expansion: Baseline up to Day 2023].

The PFS time (based on investigator assessments), according to the RECIST 1.1, was defined as the time from first administration of study treatment until first documentation of progressive disease (PD) or death when death occurred within 12 weeks of the last tumor assessment or first administration of study treatment (whichever was later). PD was defined as at least a 20% increase in the sum of longest diameter (SLD), taking as reference the smallest SLD recorded from baseline or the appearance of 1 or more new lesions and unequivocal progression of non-target lesions. The analysis of PFS was performed with a Kaplan-Meier method.

Dose Expansion Cohort: Immune Related Progression-Free Survival (irPFS) Time According to Modified Immune-Related Response Criteria (irRC) [Time Frame: Dose Expansion: Baseline up to Day 2023].

The irPFS time was defined as the time from first administration of study treatment until first documentation of immune-related progressive disease (irPD) or death when death occurred within 12 weeks of the last tumor assessment or first administration of study treatment (whichever was later). irPD: sum of the longest diameters of target and new measurable lesions increases greater than or equal to [>=]20%, confirmed by a repeat, consecutive observations at least 4 weeks from the date first documented. The analysis of irPFS will be performed with a Kaplan-Meier method. Data for immune related progression-free survival time has been reported.

Dose Expansion Cohort: Overall Survival (OS) Time [Time Frame: Dose Expansion: Baseline up to Day 2023].

Overall survival time was measured as time in months first administration of trial treatment to death. The analysis of OS time was performed with a Kaplan-Meier method.

Dose Escalation Cohort (Excluding Once Weekly Avelumab 10 mg/kg Cohort): Programmed Death Ligand 1 (PD-L1) Receptor Occupancy [Time Frame: Pre-infusion on Day 1; 48 hours after infusion on Day 3; Pre-infusion on Days 15, 43, and 85].

Percentage of PD-L1 receptors occupied by avelumab on human lymphocytes (CD3+ T-cells) was assessed by flow cytometry on peripheral blood mononuclear cell (PBMC) samples. Greater than or equal to [>=]85 percent [%] of cell viability was required for reliable receptor occupancy assessment.

Dose Expansion Cohort: Number of Participants With Positive Programmed Death Receptor-1 Ligand-1 (PD-L1) Biomarker Expression in Tumor Tissue [Time Frame: Dose Expansion: Baseline up to Day 2023].

PD-L1 assessment was performed using immunohistochemistry. PD-L1 expression status was classified as positive or negative based on the following cut-offs: For tumor cells: Participants were considered PD-L1 expression positive (negative): —if at least (less than) 5% of the tumor cells show PD-L1 membrane staining>=1+, respectively. This was used as the primary cut-off, —if at least (less than) 25% of the tumor cells show PD-L1 membrane staining>=2+, respectively. This was considered as secondary cut-off, —if at least (less than) 1% of the tumor cells show PD-L1 membrane staining>=1+, respectively. This was used as the tertiary cut-off, —if at least (less than) 50% of the tumor cells show PD-L1 membrane staining>=1+, respectively. This was used as the ‘50% cut-off’; —if at least (less than) 80% of the tumor cells show PD-L1 membrane staining>1+, respectively. This was used as the ‘80% cut-off’.

Primary Expansion Cohorts: Number of Participants With Unconfirmed Response at Week 13 According to Response Evaluation Criteria in Solid Tumors (RECIST) Version 1.1 [Time Frame: Week 13].

The response criteria evaluation was carried out according to Response Evaluation Criteria in Solid Tumors (RECIST) 1.1. CR and PR did not need to be confirmed by a subsequent tumor assessment due to blinded central assessment. CR: Disappearance of all target lesions since baseline; PR: At least a 30% decrease in the sum of the diameters of target lesions, taking as reference the baseline sum of diameters; SD: Neither sufficient increase to qualify for PD nor sufficient shrinkage to qualify for PR and PD: at least a 20% increase in SLD, taking as reference smallest SLD recorded from baseline/appearance of 1 or more new lesions and unequivocal progression of non-target lesions. Number of participants with unconfirmed response at week 13 according to response evaluation criteria in solid tumors (RECIST) version 1.1 were reported.

Dose Expansion Cohort: Duration of Response According to Response Evaluation Criteria in Solid Tumors (RECIST) Version 1.1 Per Investigator Assessment [Time Frame: Dose Expansion: Baseline up to Day 2023].

Duration of response according to RECIST 1.1, per investigator assessment was calculated for each participant with a confirmed response (complete response [CR] or partial response [PR]) as the time from the first observation of response to the first observation of documented disease progression (or death within 12 weeks of the last tumor assessment). CR: Disappearance of all evidence of target and non-target lesions. PR: At least 30 percent (%) reduction from baseline in the sum of the longest diameter (SLD) of all lesions. Results were calculated based on Kaplan-Meier estimates.

Dose Expansion Cohort: Duration of Response According to Modified Immune-Related Response Criteria (irRC) Per Investigator Assessment [Time Frame: Dose Expansion: Baseline up to Day 2023].

Duration of response according to modified irRC, per investigator assessment was calculated for each participant with a confirmed response (immune-related complete response [irCR] or immune-related partial response [irPR]) as the time from the first observation of response to the first observation of documented disease progression (or death within 12 weeks of the last tumor assessment). irCR: Complete disappearance of all tumor lesions (both index and non-index lesions with no new measurable/unmeasurable lesions). irPR: At least 30% reduction from baseline in the sum of the longest diameter (SLD) of all lesions). Results were calculated based on Kaplan-Meier estimates.

Efficacy Expansion Cohorts: Duration of Response According to Response Evaluation Criteria in Solid Tumors (RECIST) Version 1.1 as Per Independent Endpoint Review Committee (IERC) [Time Frame: Efficacy Expansion: Baseline up to Day 1072].

Duration of response according to modified irRC, per investigator assessment was calculated for each participant with a confirmed response (immune-related complete response [irCR] or immune-related partial response [irPR]) as the time from the first observation of response to the first observation of documented disease progression (or death within 12 weeks of the last tumor assessment). irCR: Complete disappearance of all tumor lesions (both index and non-index lesions with no new measurable/unmeasurable lesions). irPR: At least 30% reduction from baseline in the sum of the longest diameter (SLD) of all lesions). Results were calculated based on Kaplan-Meier estimates.

Efficacy Expansion Cohorts: Progression-Free Survival (PFS) Time According to Response Evaluation Criteria in Solid Tumors Version (RECIST) 1.1 as Per Independent Endpoint Review Committee (IERC) [Time Frame: Efficacy Expansion: Baseline up to Day 1072].

The PFS time (based on IERC), according to the RECIST 1.1, was defined as the time from first administration of study treatment until first documentation of progressive disease (PD) or death when death occurred within 12 weeks of the last tumor assessment or first administration of study treatment (whichever was later). PD was defined as at least a 20% increase in the sum of longest diameter (SLD), taking as reference the smallest SLD recorded from baseline or the appearance of 1 or more new lesions and unequivocal progression of non-target lesions. The analysis of PFS was performed with a Kaplan-Meier method.

Dose Escalation Cohort (Excluding Once Weekly Avelumab 10 mg/kg Cohort): Number of Participants With at Least 1 Positive Anti Drug Antibodies (ADA) [Time Frame: Dose Escalation: Baseline up to Day 1023].

Serum samples were analyzed by a validated electrochemiluminescence immunoassay to detect the presence of antidrug antibodies (ADA). Number of participants with ADA positive results for Avelumab were reported.

Dose Expansion Cohort: Number of Participants With At least 1 Positive Anti Drug Antibodies (ADA) Assay [Time Frame: Dose Expansion: Baseline up to Day 2023].

Serum samples were analyzed by a validated electrochemiluminescence immunoassay to detect the presence of antidrug antibodies (ADA). Number of participants with ADA positive results for Avelumab were reported.

Given the results and the example below, it is clear that treatment of subjects treated with an ICI and NK cells reduces the frequency and severity of ICI related AEs as compared to the frequency and severity of AEs in subjects treated with an ICI alone.

### Avelumab—Pembrolizumab:

In a study of 18 patients treated with avelumab (800 mg IV) and SNK01 4×10⁹ cells every other week a total of 154 doses of avelumab were delivered. Of the total doses delivered there was one grade 3 toxicity (diarrhea) and no infusion related reactions. Similar lack of toxicity was seen in a trial of 18 patients randomized to Pembrolizumab 200 mg IV+/−SNK01 in advanced NSCLC.

SNK01 appears to greatly reduce irAEs compared to historical monotherapy data while allowing for prolonged chronic administration.

The frequency of various AEs associated with ICI monotherapy and ICI+NK cells are listed in Table 6.

TABLE 6

| AEs associated | | | | Avelumab + |
|---|---|---|---|---|
| with Checkpoint | | Pembro + SNK | | SNK |
| Inhibitors | Pembro | (3 patients) | Avelumab | (17 patients) |
| Diarrhea | 19.10% | 0% | 10% | 6% |
| Colitis | 3.70% | 20% | 0% | 0% |
| Pneumonitis | 4.70% | 0% | 0% | 0% |
| Skin Adverse | 16.10% | 7% | 13% | 0% |
| Events | | | | |
| Endocrine | 23.40% | 7% | 7% | 0% |
| dysfunction | | | | |
| Hepatitis | 1.80% | 0% | 6.80% | 0% |
| Myocarditis | 0% | 0% | 0% | 0% |
| Neurotoxicity | 0% | 0% | 0% | 0% |
| Renal | 0% | 13% | 1% | 0% |
| irAEs | 69% | 47% | 16.50% | 6% |

Given the above results, it is clear that treatment of subjects treated with an ICI (Pembro or Avelumab) and NK cells reduces the frequency and severity of ICI related AEs as compared to the frequency and severity of AEs in subjects treated with an ICI alone.

irAEs by CPI and NK Cells

Although the checkpoint inhibitor (CPI) blockade of TCR inhibitory signaling pathways can successfully restore or stimulate an antitumor immune response, up to 40% of patients develop immune-related adverse events (irAE) during treatment. irAEs have a significant impact and may result in dose interruption or discontinuation, negatively affecting clinical outcomes, survival, quality of life, and can even lead to mortality. It is suggested that irAEs result from activation-based TCR signaling induced by over-elimination of inhibitory CPI receptors, which reduces peripheral immune tolerance in potentially autoreactive T cells predisposed to tissue-specific activation. As well, activated NK cells are known to recognize and kill CD4 and CD8 T cells that have been activated by antigen presenting cells dependently on NKG2D activating receptors on NK cells.

Whether SNK cells are able to kill activated T cells that are treated with OKT3 (anti-CD3) plus IL-2 or PMA plus ionomycin for 72 hours, which are well known stimuli for T cell activation, is examined in this example. If NK cells have the ability to kill activated T cells, a confirmation of which ligands for activating receptors of NK cells are involved in this killing process is performed by comparing the expression pattern of ligands for activating receptor on T cells before and after activation and then analyzing the inhibiting effect of NK cell cytotoxicity with the treatment of relevant neutralizing antibodies.

T cell activation is performed by treating T cells with 5 ug/ml OKT3+200 U/ml IL-2, 5 ug/ml OKT3+500 U/ml IL-2, or PMA+Ionomycin. Cells are left in the activating conditions for 72 hours.

Surface marker expression on T cells is the examined. Upregulation of CD69 occurs early in activation and expression of CD25 occurs late. Downregulation of CCR7 and CD62L are also observed.

A cytotoxicity assay of NK cells against activated T cells is performed. A calcein assay evaluating NK cell mediated cytotoxicity against activated T cells using calcein AM is performed.

A flow cytometric analysis is also performed. The flow cytometric assay comprises: labeling of activated T cells with Violet cell tracer, co-incubation of violet tracer labeled T cell and NK cells (4 hr), 7AAD staining, and measuring the amount of violet+/7-AAD+ positive cells.

Flow cytometric analysis for expression of ligands for NK cell activating receptors is also performed. Activated T cells are subjected to flow cytometric analysis of ligands for NK cells activating receptors (e.g., ligands for NKG2D, NCRs, etc. on T cells).

Effect of NK Cells on the Frequency of AEs in Subject's Treated with an ICI

To assess the effect of NK cells on the frequency of AEs in subjects treated with an ICI, subjects are intravenously administered 200 mg pembrolizumab and 4×10⁹ CD56+ and/or CD3−/CD56+ natural killer (NK) cells derived from peripheral blood mononuclear cells (PBMCs) to the subject. The frequency and severity of AEs and irAEs are determined both prior to and following administration of the NK cells. It is expected that administration of the NK cells will reduce the frequency of AEs related to the pembrolizumab. For example, it is expected that administration of the NK cells will reduce the frequency of diarrhea, colitis, pneumonitis, skin adverse events, endocrine dysfunction, hepatitis, myocarditis, neurotoxicity, renal adverse events, and irAEs, by up to about 100%.

In at least some of the embodiments described herein, one or more elements used in an embodiment can interchangeably be used in another embodiment unless such a replacement is not technically feasible. It will be appreciated by those skilled in the art that various other omissions, additions and modifications may be made to the methods and structures described herein without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a method having at least one of A, B, and C” would include but not be limited to methods that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a method having at least one of A, B, or C” would include but not be limited to methods that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one of skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed herein. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those of skill in the art. The various aspects and embodiments disclosed herein are for purposes of illustration and are not intended to be limiting, with the true scope and spirit being indicated by the following claims.

1. A method of reducing the likelihood of an adverse event (AE) in a subject treated with an immune checkpoint inhibitor (ICI); the method comprising:
administering an ICI to a subject, and
administering a collection of natural killer (NK) cells to the subject; wherein
NK cells are administered in an amount sufficient to reduce the likelihood and/or severity of one or more AE in the subject as compared to the likelihood and/or severity of the AE in the subject prior to administration of the NK cells.

2. A method of reducing the likelihood of an adverse event (AE) in a subject treated with an anti-PD1 or anti-PDL1 therapy; the method comprising:
administering an anti-PD1 or anti-PDL1 therapy to a subject, and
administering a collection of natural killer (NK) cells to the subject; wherein
NK cells are administered in an amount sufficient to reduce the likelihood and/or severity of one or more AE in the subject as compared to the likelihood and/or severity of the AE in the subject prior to administration of the NK cells.

3. A method of reducing the likelihood of an adverse event (AE) in a subject treated with pembrolizumab; the method comprising:
administering pembrolizumab to a subject, and
administering activated and expanded CD3−/CD56+ natural killer (NK) cells derived from peripheral blood mononuclear cells (PBMCs) to the subject; wherein
the NK cells are administered in an amount sufficient to reduce the likelihood and/or severity of one or more AE in the subject as compared to the likelihood and/or severity of the AE in the subject prior to administration of the NK cells.

4. A method of reducing the likelihood of an adverse event (AE) in a subject treated with pembrolizumab; the method comprising:
intravenously administering 200 mg pembrolizumab to a subject, and
administering 4×10⁹ activated and expanded CD3−/CD56+ natural killer (NK) cells derived from peripheral blood mononuclear cells (PBMCs) to the subject; wherein
administration of the NK cells reduces the likelihood and/or severity of one or more AE in the subject as compared to the likelihood and/or severity of the AE in the subject prior to administration of the NK cells.

5. The method of claim 1, wherein the NK cells are activated and expanded CD3−/CD56+ NK cells derived from peripheral blood mononuclear cells (PBMCs).

6. The method of claim 1, wherein the ICI comprises a PD-1 or PD-L1 inhibitor.

7. The method of claim 1, wherein the ICI is an ICI selected from the list comprising: pembrolizumab, nivolumab, atezolizomab, durvalumab, avelumab, camrelizumab, cemiplimab, sintilimab, tisleilizumab, toripalimab, lpilimumab, lpilimumab plus nivolumab, or tremelimumab.

8. The method of claim 1, wherein the ICI is pembrolizumab.

9. The method of claim 1, wherein between about 1×10⁹−9×10⁹ CD3−/CD56+ NK cells are administered to the subject.

10. The method of claim 1, wherein administration of the NK cells decreases the frequency of diarrhea, colitis, pneumonitis, skin adverse event, endocrine dysfunction, hepatitis, myocarditis, neurotoxicity, renal adverse events, irAEs, and/or any combination thereof, by up to 100% as compared to the frequency of diarrhea, colitis, pneumonitis, skin adverse event, endocrine dysfunction, hepatitis, myocarditis, neurotoxicity, renal adverse events, irAEs, and/or any combination thereof, prior to administration of the NK cells.

11. The method of claim 1, wherein administration of about 4×10⁹ CD3−/CD56+ NK cells decreases the frequency of diarrhea, colitis, pneumonitis, skin adverse event, endocrine dysfunction, hepatitis, myocarditis, neurotoxicity, renal adverse events, irAEs, and/or any combination thereof, in a subject treated with 200 mg pembrolizumab by up to 100% as compared to the frequency of diarrhea, colitis, pneumonitis, skin adverse event, endocrine dysfunction, hepatitis, myocarditis, neurotoxicity, renal adverse events, irAEs, and/or any combination thereof, prior to administration of the CD3−/CD56+ NK cells.

12. The method of claim 1, wherein the ICI and NK cells are administered at the same time.

13. The method of claim 1, wherein the ICI is administered prior to the NK cells.

14. The method of claim 1, wherein the NK cells are administered prior to the ICI.

15. The method of claim 6, wherein the anti-PD1 or anti-PDL1 inhibitor is an antibody that binds to PD-1 or PDL1.

16. The method of claim 8, wherein 200 mg of the pembrolizumab is administered.

17. The method of claim 16, wherein the pembrolizumab is administered intravenously.

18. The method of claim 8, wherein the pembrolizumab is administered together with 4×10⁹ activated and expanded CD3−/CD56+ natural killer (NK) cells derived from peripheral blood mononuclear cells (PBMCs).

19. The method of claim 1, wherein administering a collection of natural killer (NK) cells to the subject comprises administering about 4×10⁹ CD3−/CD56+ cells.
